(19) United States
(12) Patent Application Publication
(10) Pub. No.: US 2024/0165879 A1
(43) Pub. Date: May 23, 2024

(54) MULTI-MATERIAL BIOPRINTING

(71) Applicant: University of Connecticut, Farmington, CT (US)

(72) Inventors: Ali TAMAYOL, Storrs, CT (US); Mohamadmahdi Samandari, Storrs, CT (US)

(21) Appl. No.: 18/283,251

(22) PCT Filed: Mar. 25, 2022
(86) PCT No.: PCT/US2022/021938

§ 371 Date: Sep. 21, 2023

Related U.S. Application Data
(60) Provisional application No. 63/166,572, filed on Mar. 26, 2021.

(51) Int. Cl. B29C 64/336; A23P 20/20; A23P 20/25; B01F 25/4314; B29C 64/118; B29C 64/209; B29C 64/314; B29C 64/40; B29K 105/00; B33Y 10/00; B33Y 30/00; B33Y 40/10; B33Y 70/00

(52) U.S. Cl. CPC B29C 64/336; B01F 25/43141; B29C 64/118; B29C 64/209; B29C 64/314; B29C 64/40; B33Y 10/00; B33Y 30/00; B33Y 40/10; B33Y 70/00; A23P 20/20; A23P 2020/253; B29K 2105/0061; B29K 2995/0056

(57) ABSTRACT

A system for printing biomaterials can include a mixer having a longitudinal axis. The mixer can define a flow channel that extends along the longitudinal axis. The mixer can have at least one inlet configured to receive a first printable biomaterial and a second printable biomaterial and an outlet spaced from the inlet along the longitudinal axis. One or more mixing elements positioned within the flow channel between the inlet(s) and the outlet of the mixer. The mixing element(s) can be configured to control a spatial distribution of the first and second printable biomaterials across and along the longitudinal axis.

### CROSS-REFERENCE TO RELATED APPLICATIONS

This application claims priority to and the benefit of the filing date of U.S. Provisional Patent Application No. 63/166,572, filed Mar. 26, 2021, the entirety of which is hereby incorporated by reference herein.

### FIELD

This disclosure relates to bioprinting and, in particular to multi-material bioprinters and methods of use of such multi-material bioprinters as well as materials printed by such multi-material bioprinters.

### BACKGROUND

The biofabrication of living constructs is critical for regenerative medicine, in vitro tissue modeling, food biomanufacturing, etc. Bioprinting is a promising method for biofabrication of living constructs. Conventional bioprinting methods are limited to deposition of single material at a time. However, native tissues typically involve multiple compartments including different cell types, extracellular matrices, and vascular and neural networks. Recapitulating such complex tissues using bioprinting require multiple materials, cells, regenerative factors, chemical and physical properties, and different architectures to be integrated with each other at a biomimetic resolution. A notable example is the need for microcapillaries for transport of nutrients deep into the tissue constructs, which can be addressed by bioprinting of multicompartmental filaments consisting of sacrificial compartments forming capillaries upon dissociation.

The biomimetic resolution of the bioprinted constructs is not only required for proper function of the constructed tissue, but also is critical during the development and maturation of the target tissue. As an example, cellular organization can be controlled with physical and chemical cues in their environment, which subsequently affect their behavior such as proliferation, differentiation and maturation. While current chemical and topological patterning methods have been shown to successfully fabricate miniaturized tissue models, they suffer from significant limitations such as setup complexity, the negative impact of external fields on cells, limited construct size, multistep fabrication processes, and low throughput, presenting significant challenges for their clinical translation and application in food industries. Furthermore, current bioprinting approach fail to form living constructs with biomimetic multicompartmental architecture and resolution.

Existing bioprinting technologies fail to deliver spatial control of multiple materials with high accuracy, which is usually required for regenerative medicine and in vitro tissue modeling, and high throughput, which is crucial for food biomanufacturing. The resolution of current extrusion-based printers is not high enough for many tissue engineering applications. Current limitations on resolution prevent control over the features within each extruded filament to the extent needed to recapitulate physiologically detailed features like vascular, neural, and lymph tissues. As further disclosed herein, the described bioprinting systems and methods can address these and other deficiencies of existing technologies.

### SUMMARY

Described herein, in various aspects, is a system comprising: a mixer having a longitudinal axis, the mixer defining a flow channel that extends along the longitudinal axis. The mixer can comprise at least one inlet configured to receive a first printable biomaterial and a second printable biomaterial and an outlet spaced from the inlet along the longitudinal axis. At least one mixing element (e.g., at least one static mixing element) can be positioned within the flow channel between the at least one inlet and the outlet of the mixer. The at least one mixing element can be configured to control a spatial distribution of the first and second printable biomaterials across and along the longitudinal axis.

Additional advantages of the disclosed bioprinter and bioprinting systems and methods will be set forth in part in the description that follows, and in part will be obvious from the description, or may be learned by practice of the claimed invention. The advantages of the disclosed bioprinter and bioprinting systems and methods will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended drawings wherein:

FIG. 1A is a schematic perspective view of a multicompartmental bioprinting system for forming microcompartmental hydrogel fibers as disclosed herein, showing a connector, delivering different biomaterial precursors to a mixing module forming striations of different hydrogels with predesigned spatial distribution, connecting to a nozzle, and integrated with a coaxial microfluidic device to extrude the mixed streams of biomaterial precursors through a sheath flow and form multicompartmental fibers. FIG. 1B illustrates a front portion of the printer of FIG. 1 that defines a Luer lock coupling. FIG. 1C is a schematic diagram of a mixer of the bioprinter of FIG. 1. FIG. 1D shows cross sections of different material arrangements formed by the mixer in the plane C-C of FIG. 1C. FIG. 1E is a schematic diagram of a nozzle of the bioprinter of FIG. 1. FIG. 1F shows different exemplary cross sections of the nozzle in the plane e-e of FIG. 1E. FIG. 1G shows a schematic diagram of a nozzle and a sleeve forming a sleeve channel.

FIG. 2 illustrates fluid dynamics simulations demonstrating that increasing the resolution of the multimaterial bioprinting using an exemplary mixing modules (KENICS mixer) does not significantly increase the shear stress and required extrusion pressure. The shear stress (t) map in the (i) static mixer and (ii) conical nozzles with different tip diameters. The tip diameter (D) was reduced by half in different simulations, corresponding to the application of each static mixer element subdividing the upper stream into two half-sized sub-streams. The shear stress across the nozzle tips relative to the maximum shear stress in the static mixer (τₘₐₓ) is graphed (iii). The pressure (P) map in the (iv) static mixer and (v) conical nozzles with different tip diameters. The pressure inside the conical nozzles relative to the maximum pressure inside the static mixer (Pmax) is graphed (vi).

FIG. 3 shows a side view of an exemplary static mixer as disclosed herein.

FIG. 4 shows mixing of inks (e.g., hydrogels) within the static mixer of FIG. 3.

FIG. 5 shows simulation and corresponding experimental cross sections of flow exiting the static mixer of FIG. 4, taken in the plane b-b, for mixers with different numbers of mixer elements.

FIG. 6 shows phase contrast images of multicompartmental alginate/GelMA fibers demonstrating the fiber's internal microfilaments for five mixer elements, seven mixer elements, and fiber formed from premixed inks.

FIG. 7A illustrates a plot of a ratio of radius of the fiber to the radius of the inner channel versus the ratio of flow rate of the outer channel to the flow rate of the inner channel, illustrating control over the fiber diameter using the coaxial microfluidic device. FIG. 7B illustrates flow profiles for different ratios of flow rate of the outer channel to the flow rate of the inner channel. shows simulation results demonstrating the effect of varying flow of the inner and outer streams of the system for forming microcompartmental hydrogel fibers.

FIG. 8 illustrates the formation of angled microcompartments inside the hydrogel fiber experimentally and numerically.

FIG. 9A shows an F-Actin/DAPI staining demonstrating the alignment of myoblasts along the axis of an exemplary fiber, 24 hours post-encapsulation. FIG. 9B shows an F-Actin/DAPI staining demonstrating the cells could not align in normal hydrogel fiber formed from pre-mixed hybrid hydrogel solution.

FIG. 10 illustrates plots showing quantitative evaluation of F-actin cytoskeleton (left) and nuclei (right) directionality within multicompartmental hydrogel fibers (MCHFs) compared with hydrogel fibers fabricated from pre-mixed bioink.

FIG. 11 shows a plot of metabolic activity over time, illustrating enhanced metabolic activity of the cells cultured in MCHFs compared to the cells cultured in pre-mixed fibers. n=4 for each time point. Scale bars are 500 μm for the top row and 200 μm for the bottom magnified images.

FIG. 12A comparisons of simulations and cellular grown between different outer and inner flow rates of fibers printed using the system of FIG. 1, demonstrating the ability to control the angle of internal microcompartments to modulate the cellular alignment using experiments and numerical simulations. FIG. 12B shows a plot of a distribution of F-actin orientation at different ratio of flow rates, corresponding to the images shown in FIG. 12A. A static mixer with five mixing elements was used for formation of internal microfilaments.

FIG. 13A shows side views (top) and magnified side views (bottom) for days 1, 3, 5, and 7 (left-to-right) of encapsulated myoblasts in MCHFs. FIG. 13B shows myogenic progression of the cells in hydrogel fibers using gene expression analysis with RT-qPCR.

FIG. 14 shows views of a multilayered mesh (top), and unidirectional structure (middle) formed from fibers as disclosed herein. The magnified view below confirms preservation of the internal microfilaments generated by a static mixer upon printing.

FIG. 15 illustrates different biotextile techniques including (i) weaving, (ii) braiding, (iii) knotting, and (iv) coil formation for combining MCHFs as disclosed herein.

FIG. 16 shows cell-laden constructs fabricated through bio-textile assembly of MCHFs. Actin-DAPI assay was used for staining.

FIG. 17A shows a schematic representation of the fabrication process. FIG. 17B shows scanning electron micrographs (SEM) images of filaments printed using 3, 4, 5, or 6 helical mixing elements and alginate (permanent ink) and pluronic F-127 (fugitive ink). Scale bar: 200 μm. FIG. 17C is a plot showing the channel width decreases with increasing numbers of helical mixing elements in the printhead.

FIG. 18A illustrates an example of a multi-material filament that is composed of a sacrificial gelatin core and GelMA outer structure printed by the bioprinter. FIG. 18B illustrates the cross sections of the printed fiber from FIG. 18A forming channels therein.

FIG. 19 is an image of an exemplary static mixer for low speed multicompartmental bioprinting.

FIG. 20A illustrates a Christmas-tree gradient generator-style mixer. FIG. 20B shows a side view of the filament, demonstrating the ability of the mixer of FIG. 20A to form a continuous gradient of differently colored precursor materials across the cross-section of the extruded fiber. FIG. 20C illustrates pixel intensity quantification across the length of the fiber to demonstrate the continuous gradient.

FIG. 21A illustrates a mixer for forming a fiber. FIG. 21B depicts an exemplary profile of a fiber formed by the mixer shown in FIG. 21A.

FIG. 22 shows an operating environment including an exemplary configuration of the computing device that can be used with the exemplary bioprinter as disclosed herein.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a,” “an,” and “the” include plural referents unless the context clearly dictates otherwise. For example, use of the term “a helical mixing element” can refer to one or more of such helical mixing elements, and so forth.

All technical and scientific terms used herein have the same meaning as commonly understood to one of ordinary skill in the art to which this invention belongs unless clearly indicated otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedent “about,” it is contemplated that values within up to 15%, up to 10%, up to 5%, or up to 1% (above or below) of the particularly stated value can be included within the scope of those aspects. Similarly, in some optional aspects, when values are approximated by use of the terms “substantially” or “generally,” it is contemplated that values within up to 15%, up to 10%, up to 5%, or up to 1% (above or below) of the particular value can be included within the scope of those aspects. When used with respect to an identified property or circumstance, “substantially” or “generally” can refer to a degree of deviation that is sufficiently small so as to not measurably detract from the identified property or circumstance, and the exact degree of deviation allowable may in some cases depend on the specific context.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of” For example, “at least one of A, B and C” explicitly includes only A, only B, only C, and combinations of each.

The word “or” as used herein means any one member of a particular list and, unless context dictates otherwise, can also include any combination of members of that list.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

Engineering of large tissues for regenerative medicine and food biomanufacturing require biofabrication of constructs involving multiple compartments including various cell types, extracellular matrix, vascular and neural networks, etc. Therefore, a proper biofabrication process should address the requirement of constructing complex multi-tissues with biomimetic resolution. Furthermore, cells in most tissues exhibit a high level of organization in their spatial distribution and alignment. This organized architecture can be advantageous or even critical to proper cellular development during maturation and the function of the mature tissue. Therefore, biofabricated cellular scaffolds for tissue engineering applications can mimic this native architecture of the tissue to reproduce the behaviors of natural tissue.

Fiber-based biofabrication techniques have been implemented for engineering various tissues. Ranging from extrusion bioprinting to biotextile processes, fiber-based tissue engineering has been employed as a high-throughput, simple, and cost-effective method for assembly of cell-laden fibers to 3D complex constructs. These fibers can act as the building blocks of biomimetic constructs for engineering muscle. Adjustable mechanical properties and control over geometry and composition of final structure are distinct advantages of fiber-based approaches in the context of tissue engineering. However, creating biomimetic architecture of the tissues involving various cell types, cell scaffolding materials, vasculature and neural network with highly ordered cellular organization within the individual fibers of such constructs has proven challenging, since the dimensions of fibers compatible with biotextile processes and extrusion bioprinting are much larger than cell-scale sizes, reducing the boundary effects on cellular organization.

Disclosed herein are systems and methods for multicompartmental bioprinting of complex tissues with biomimetic resolution. This method is creating compartmentalized fibers with controllable internal architecture. While controlling the fiber diameter in larger scales, the size of each compartment can also be easily tuned down to dimensions recognizable by cells to allow effective direction of cellular alignment within the fiber. To demonstrate the potential of the strategy, the effect of this biofabricated architecture on muscle cell growth, morphology, and function are shown. This strategy can be easily applied to various fiber-based tissue engineering approaches, including 3D bioprinting and biotextile manufacturing, to form complex multi-tissues with biomimetic resolution and control cellular organization.

Multicompartmental printing can be highly crucial for the proper treatment of volumetric defects in various tissues or in cases of polytrauma where multiple tissues are injured. Further, this since the disclosed herein multicompartmental bioprinting approach is a high throughput method that can enable rapid cellular maturation, it is an excellent candidate for food biomanufacturing through cellular agriculture, followed by assembly of the fibers with bioprinting or biotextile process. This method is capable of printing different materials with biomimetic resolution to recapitulate native tissue architecture.

The use (and printing) of multiple materials enables the construction of physiologically relevant tissues. The spatial control over the composition of the depositing bio-ink both across and along the filament, offered by multicompartmental bioprinting disclosed herein, provides the ability to recreate more complex structures and tissues than are available with currently available extrusion or material jetting additive manufacturing (3D printing) systems. Thus, the disclosed systems and methods allow for controlling the physical and chemical properties of the scaffold with resolution comparable to native tissue structures, to better tune the scaffold for enhanced maturation. The ability to integrate multiple materials and refine the resolution of bioprinting can save money and increase the types of tissues that can be engineered for regenerative medicine, in vitro tissue modeling, and food biomanufacturing. Furthermore, controlling the interaction between different materials to be printed and their response to environmental conditions, such as in vivo physical and chemical stimuli, in such sub-filament resolution further enables the formation of scaffolds beyond the capability of other bioprinters. Recreating the interface between various tissues requires creating precise gradients at short length scales and that can only be achievable by having sub-filament resolution in a way that the position of multiple materials and their concentrations can be controlled at the nozzle. The disclosed bioprinting systems and methods can take advantage of physical and chemical cues generated inside the filaments for an improved regeneration rate and an improved quality of regeneration in regenerative medicine, as well as enhancing the efficiency and versatility of the cellular agriculture in food biomanufacturing.

The multicompartmental bioprinting can allow for the fabrication of scaffolds. In some aspects, the scaffolds can comprise one or more compartments. Such compartments can optionally carry distinct materials, biological factors, and/or cells. Embodiments disclosed herein can be used to print multiple materials from a single device; form fibers with multiple distinct compartments; and/or fabricate scaffolds in which every single fiber has embedded capillaries (e.g., microcapillaries) with adjustable size.

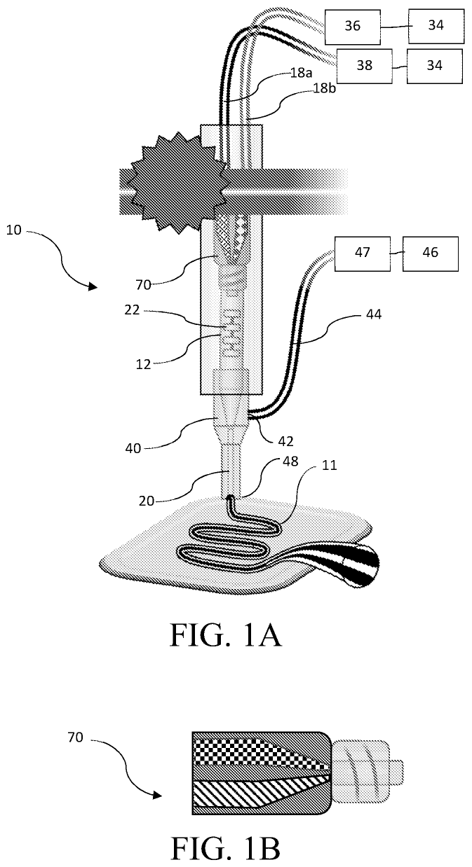

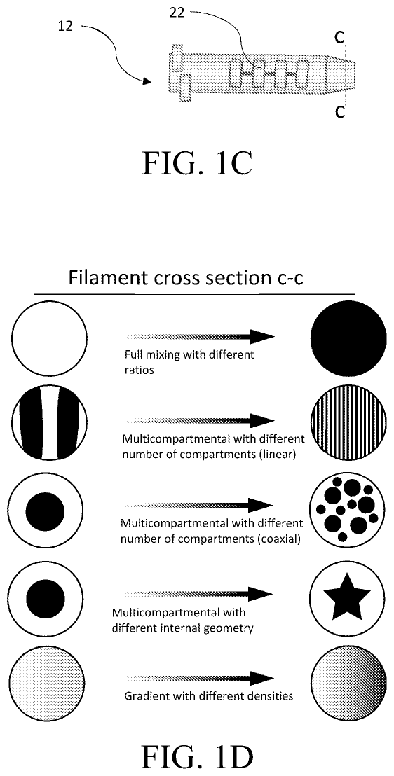

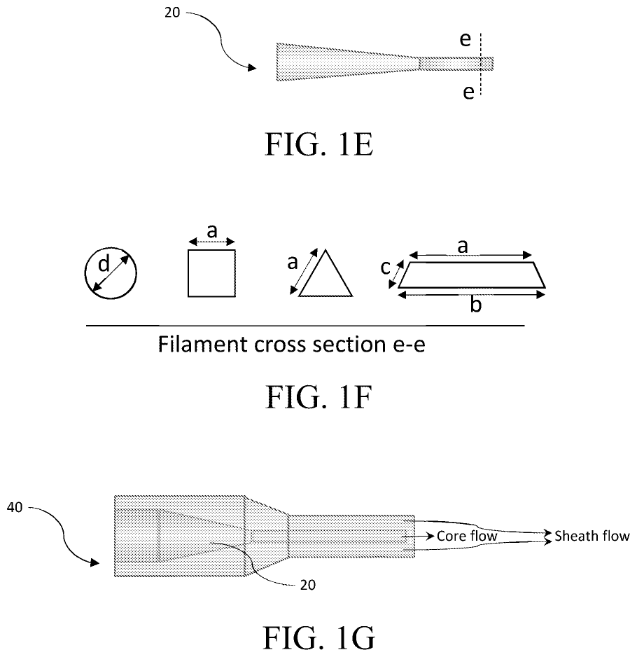

Disclosed herein and with reference to FIGS. 1-4 is a system 10 for forming fibers 11 with controlled spatial distribution of materials along a central axis of the fiber and/or across (e.g., transverse to) the fiber (FIG. 1D) as disclosed herein. In further aspects, the multicompartmental printing system 10 be configured to print multicompartmental droplets. The multicompartmental printing system 10 can be used to fabricate scaffolds with sub-filament features. These sub-filament features can be formed by one or more different material concentrations, a different number of linear compartments, a different number of coaxial compartments, varying internal geometries, or different discrete or continuous gradients. The sub-filament control over material position and architecture can enable the formation of physiologically relevant tissue-like scaffolds. After printing, some materials can be dissolved (or otherwise removed) to create sub-filament compartments. In some aspects, the scaffolds can be formed with one or more compartments therein. The compartments can serve as topological cues for cells. For example, the compartments can define architectural features to allow for the recapitulation of lymph, nervous, or vascular networks, different tissue layers, etc. In exemplary aspects, a filament or filament subunit (e.g., drop) can be configured to form linear and/or radial compartments.

The system 10 can comprise a fluidic connector 70 (FIG. 1B) delivering different biomaterials to a mixer 12 (FIG. 1C). The mixer 12 can have a longitudinal axis 14. The mixer 12 can comprise predesigned internal mixing elements 22 to form a multicompartmental fiber 11. The mixer 12 can comprise a nozzle 20 (FIG. 1E) having an outlet 21 with predesigned outlet profile (FIG. 1F), and a sleeve 40 (FIG. 1G) that is configured to guide a sheath flow of additional biomaterials or crosslinking agents. The multicompartmental bioprinting system 10 can be easily integrated with syringe pumps to independently control the flow of the biomaterials into different inlets 18 of the system (inlets of the biomaterials can be one, two, three, four, or more) and extrude different materials at a regulated rate. Flow in the sheath channel in the sleeve 40 can also be controlled independently through an inlet 42. The flowrates and positioning of the fiber deposition can also be controlled simply by integrating the multicompartmental bioprinting system with various printers including stationary, handheld, and robotic arm printers. The system 10 can deposit the fibers and stack them directly on surfaces, or spin the fiber in crosslinking bath to form individual multicompartmental fibers.

Optionally, a relative flow rate of the first material to the flow rate of the second or more materials can be varied to vary a ratio of the first material to the second or more materials along the length of a printed filament.

Different biomaterials (precursors) can be extruded into the mixer 12 and mixed to varying levels or in various arrangements to form various architectures (FIG. 1D). The mixer 20 can be provided as an adapter (e.g., a separate component that is coupled to the connector 70). Thus, a particular mixer can be selected based on a desired mixing profile and can be coupled to the connector to adapt the printer for a particular application. The mixer can be configured to control the degree of mixing and spatial organization of precursor materials across the printing filament (FIG. 1D). Modulation of the flow rate and integration of the mixing module can further allow real-time control of filament deposition along with the fiber. The nozzle 20 can control the applied stress to the material and shape of the filament upon deposition.

The nozzle 20 of the printer can be optimized for use with the polymer (or combination of polymers) being used. Optionally, the nozzle 20 can be embodied by a standard needle and tapered nozzle with any suitable gauge size. In further aspects, the nozzle can be custom-built with an inlet (e.g., a circular inlet) and any desired outlet profile (FIG. 1F). The outlet of the nozzle can have a cross section that defines the shape of the outer perimeter of a filament printed by the multicompartmental bioprinting system 10. For example, the outlet can have a cross sectional profile of a circle, oval, square, rectangle, trapezoid, triangle, or any shape or any array of shapes. The nozzle 20 can optionally define a taper toward the outlet 21. Different filament profiles can be used in the printing of scaffolds with different resolutions, volumes, and cross-sectional shapes. For example, for a burn wound which usually involves a large skin area, rapid printing of sheets covering the wound is required, while for treatment of volumetric muscle loss, a circular profile with good resolution is necessary to recapitulate the native muscle structure and fill the complex irregular-shape cavity. For food industries, different outlet profiles can be used to form noodles, sheets, stakes, etc.

The multicompartmental bioprinting system 10 can comprise a mixer 12 (e.g., a static mixer). The mixer 12 can be configured to shape the first printable material and the second printable material (and any further printable materials). For example, referring also to FIG. 1D, the mixer 12 can (optionally, with further independent control of the flow rates) control output ratios of the first printable material to the second printable material (and any further printable materials) to adjust the material composition. Still further, as further described herein, the first and second printable materials can be printed to form one or more compartments (linear and/or coaxial compartments) within a filament, and the mixer 12 can, at least in part, determine the number of compartments across the filament. For example, a first exemplary cross-sectional profile can have a pair of linear compartments. A second exemplary cross-sectional profile can have several linear compartments. A third exemplary cross-sectional profile can have a single circular compartment. A fourth exemplary cross-sectional profile can have a plurality of circular compartments. In further aspects, the mixer 12 can be configured to mix the first and second printable materials (and any further printable materials). Optionally, the mixer 12 can be configured to mix the first and second printable materials (and any further printable materials) at a ratio that changes (or respective ratios that change) across a cross section of the material as it leaves the nozzle 20.

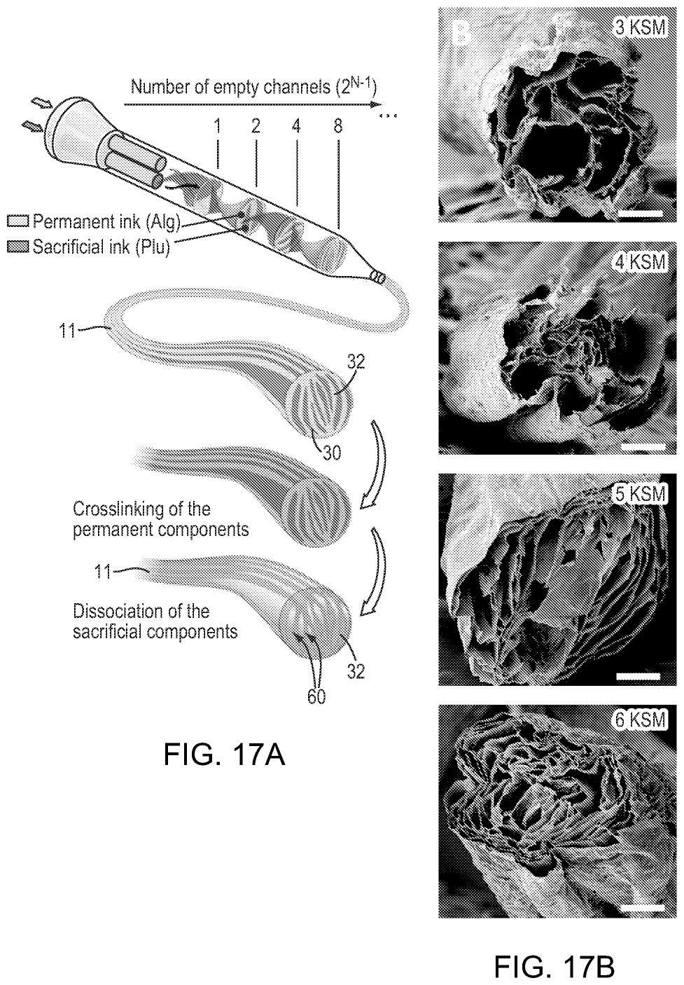

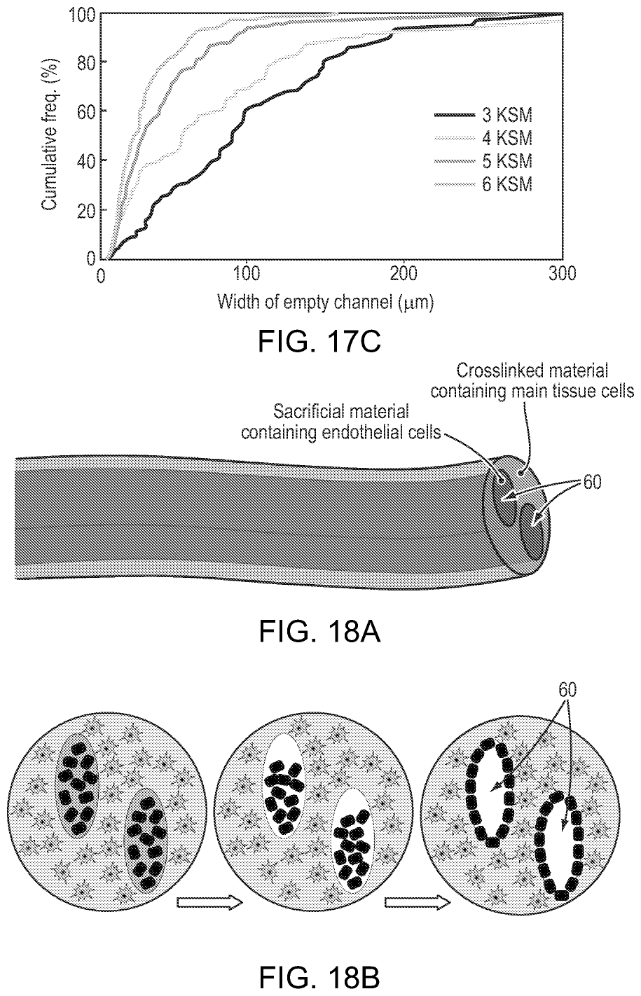

In some aspects, the mixer can be used to fabricate fibers from single or multiple materials with designed architectures. Each material can carry different components such as cells, particles, and/or chemical reagents. The materials can interact with each other upon interfacing, or be affected by external stimuli upon printing, inducing a chemical or physical change in the filament. A notable example is the use of sacrificial materials (e.g., materials that can be removed as further disclosed herein) as one of the components, which enables formation of fibers with embedded capillaries (e.g., microcapillaries). In exemplary aspects (FIGS. 17-18), the filament can have an axis of elongation, and the capillaries can have cross-sectional dimensions (e.g., a diameter) transverse to the filament axis of elongation that can range from nanometers to micrometers (such capillaries being referred to herein as “microcapillaries”). For example, the capillaries can have cross sectional dimensions from about 10 nm to about 100 micrometers, or from about 50 nm to about 50 micrometers, or from about 100 nm to about 10 micrometers.

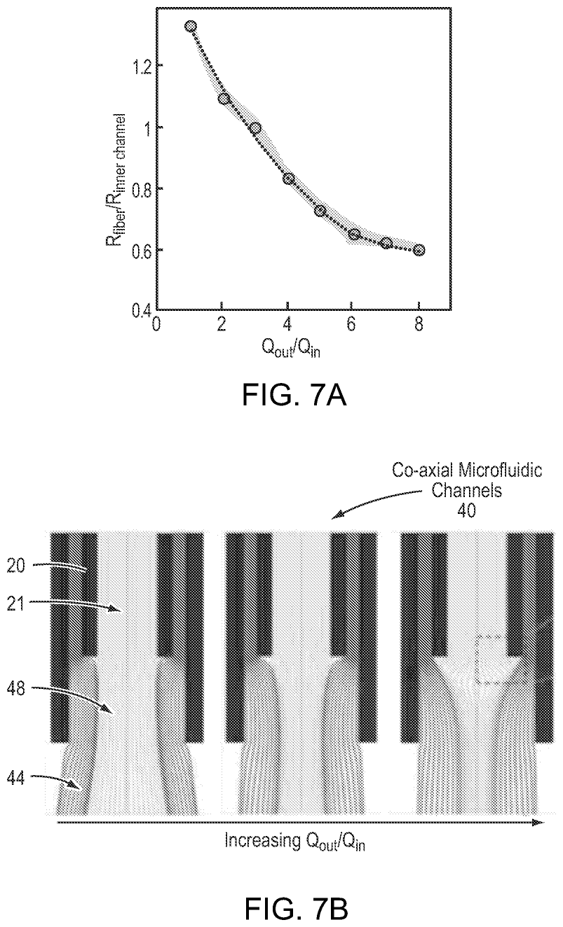

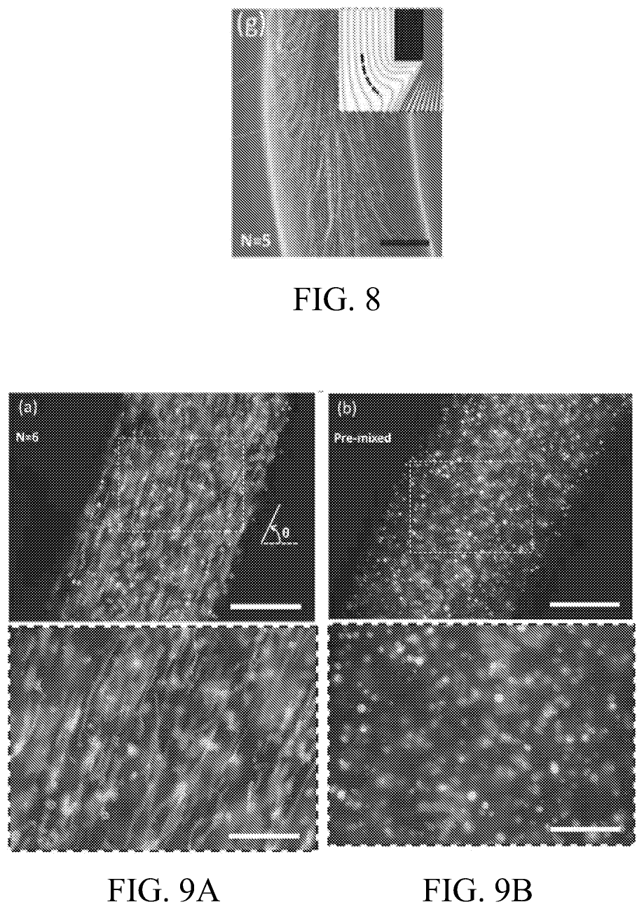

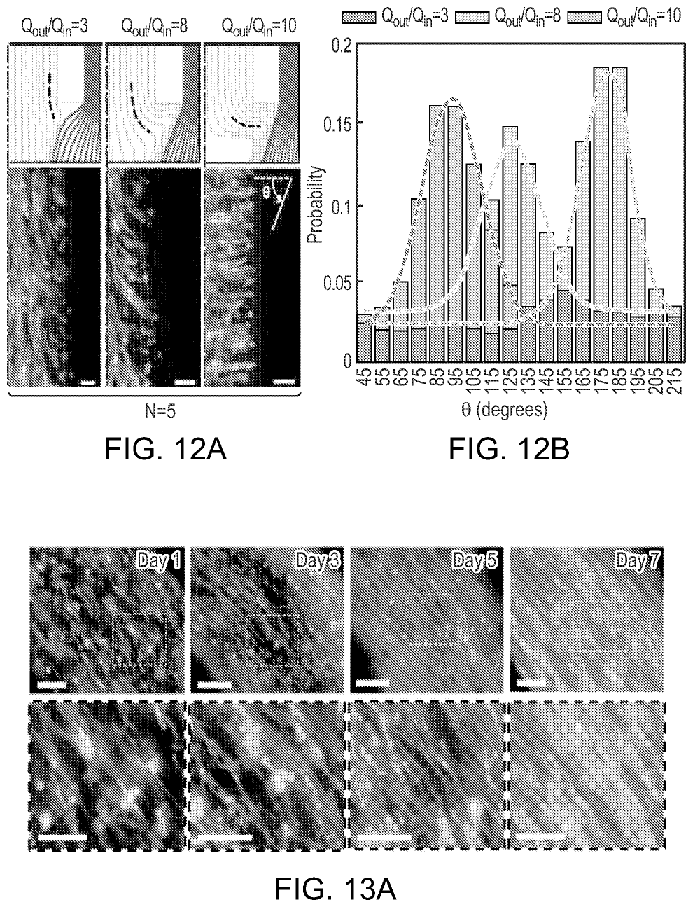

In some aspects, the system 10 can comprise a sleeve 40 (e.g., a coaxial sleeve). The sleeve can guide a sheath flow of an additional biomaterial to be incorporated into a printed fiber. The sleeve 40 can further guide a sheath flow of a crosslinking agent. The crosslinking agent can solidify one or more of the compartmental biomaterials delivering from the mixer 12 through chemical, ionic, enzymatic or other crosslinking mechanism. With reference to FIG. 7, the sleeve 40 can also control the dimension of the cross-sectional fiber 11 by tuning the relative flow rate of the sheath to core flow. With reference to FIGS. 8 and 12, the sleeve 40 can further control the organization of the internal compartments 60 of the fiber 11 by controlling the streamlines of the core flow when adjusting the relative flow rate of the sheath flow to core flow.

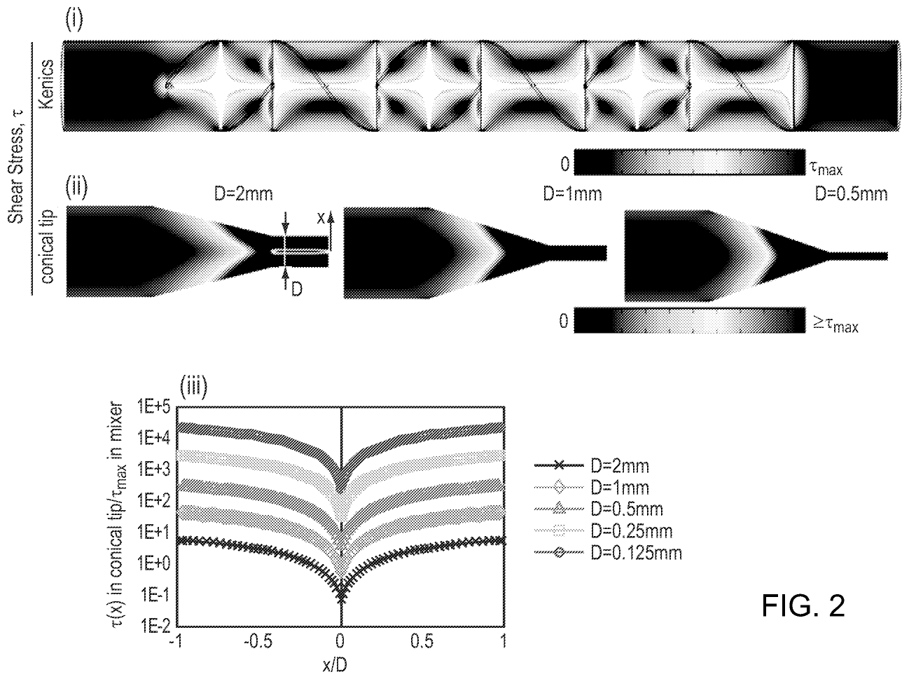

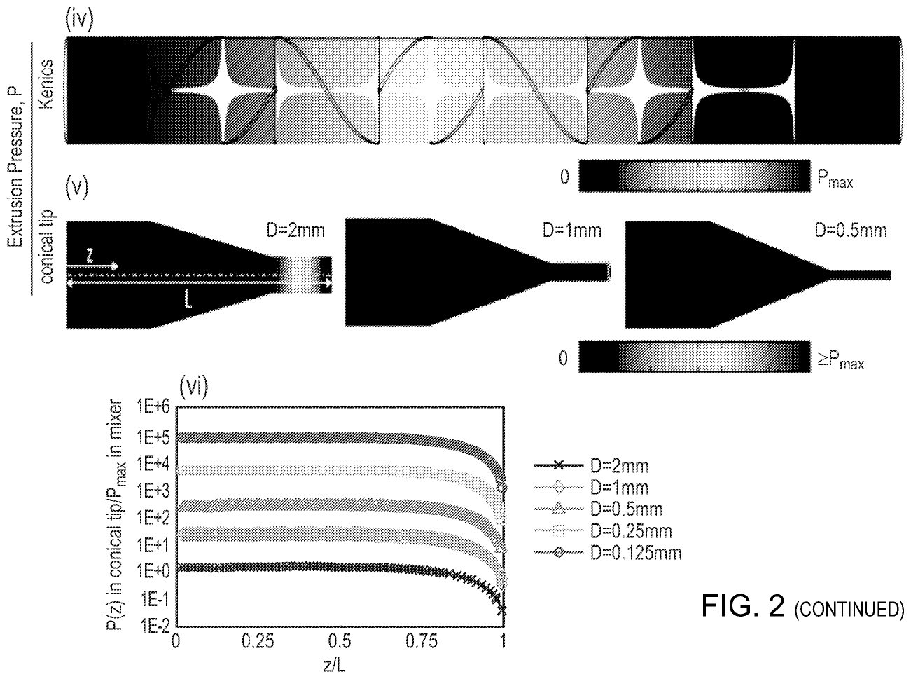

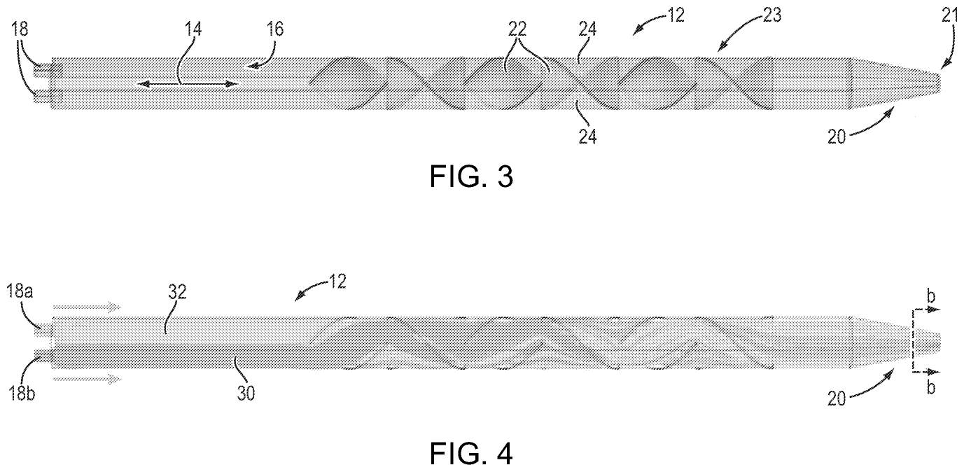

In exemplary aspects, with reference to FIGS. 2-6, the mixer 12 can comprise one or more mixing elements 22 (e.g., static mixing elements such as helical mixing elements) that are configured to divide the flow of material into two streams, rotate the streams, and interface said two streams in reverse order, as further described herein. Optionally, the mixing elements (e.g., helical mixing elements) can have a generally helicoid shape (e.g., a flat material with an intermediate 180 degree twist between opposite ends of the material). The mixing elements 22 (e.g., helical mixing elements) can be axially spaced and rotationally offset from each other (e.g., with sequential mixing elements 22 being rotationally offset by 90 degrees or about 90 degrees as shown in FIG. 3).

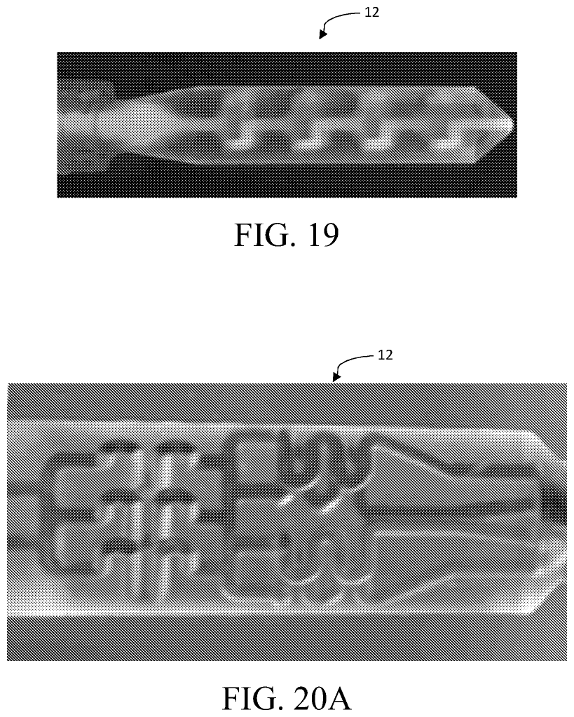

To form a multicompartmental filament with an intercalated linear (e.g., striated) structure, as shown in FIG. 4-6, varying mixing modules with varying internal geometries can be implemented. For the printing of high-viscosity materials at high flow rates, a mixer with a designed number of internal (optionally, helical) elements can be used. Optionally, the mixer can comprise or be adapted from a KINEX mixer made by Chemineer of Dayton, OH. Each internal (optionally, helical) element can divide the streams of different materials into two streams, rotate the streams and interface the streams together in reverse order. In this way, an elongated array of different material chambers can be created in the cross-section of the stream, forming a multicompartmental precursor stream. The number of the compartments (x) and their size can be controlled by the number of the implemented mixing elements (N) with x=2^{N}. Thus, a mixer 12 with a particular number of mixing elements (e.g., helical mixing elements) can be coupled to the connector 70 to form the desired number of compartments. In embodiments in which the viscosity of at least one material or the printing rate is low, to generate an intercalated linear structure, the mixer can comprise, instead of mixing elements (e.g., helical mixing elements) that divide flow evenly, a set of microfluidic channels that split the flow into two streams with different cross sectional areas and interface the two streams in the reverse order (as shown in FIG. 19).

In some aspects, the mixer 12 can comprise a plurality of mixing elements 22 (e.g., helical mixing elements) that are arranged along the longitudinal axis 14 of the mixer. In some aspects, mixer 12 can comprise from three to eight mixing elements 22 (e.g., three to eight helical mixing elements). In exemplary aspects, the mixer 12 can comprise 1, 2, 3, 4, 5, 6, 7, 8, 9, 10, or more mixing elements 22 (e.g., 1, 2, 3, 4, 5, 6, 7, 8, 9, 10, or more helical mixing elements).

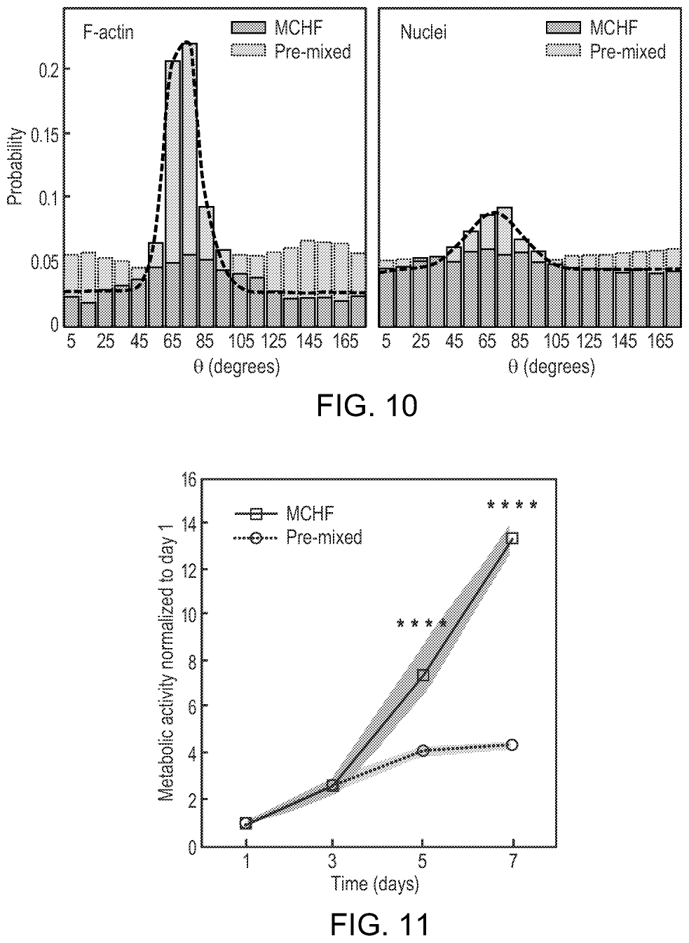

Referring to FIGS. 10A and 10B, in order to form filaments with single- or multi-encapsulated compartments, the mixer 12 can comprise a first channel 80 that flows a first material therethrough and a respective outlet 82 of the second material (and additional materials) within the first channel for each encapsulated compartment positioned within the first flow channel. The outlet(s) can be circular for forming circular compartments 84a. Optionally, a plurality of mixers can be placed in series to increase the number of internal compartments. To form coaxial filaments with different internal geometry (FIG. 1D), a specific fluidic system can be implemented with coaxial channels in which the outlet(s) 82 have the desired shape while the first channel 80 has the cross-sectional shape of the nozzle outlet (e.g., circular).

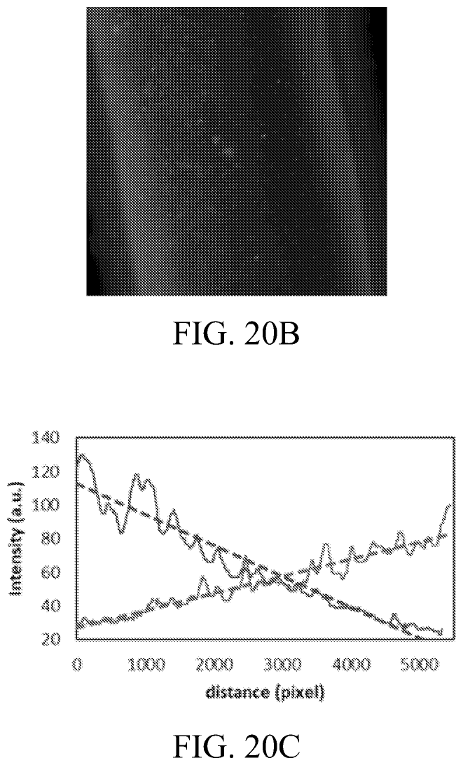

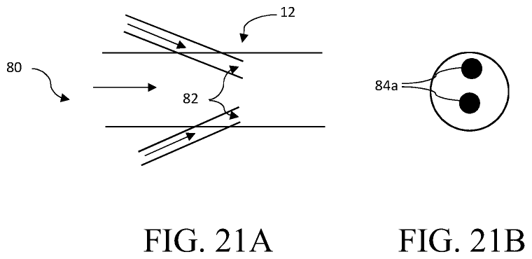

To form a gradient of materials across the filament (in cross section, as shown in FIG. 1D), a Christmas-tree gradient generator can be used (FIG. 20). For example, in some aspects, the streams of different materials are split into two streams, and one stream from each material is interfaced and thoroughly mixed, while another of each material stream is kept unmixed to form minimum and maximum concentrations. The streams can then be combined side-by side. Division and recombination can be repeated in different stages of the Christmas-tree design to form a smooth gradient across the filament. FIG. 20B shows a side view of the filament, demonstrating the ability of the microfluidic mixing chamber to form a continuous gradient of differently colored precursor materials across the cross-section of the extruded fiber. FIG. 20C illustrates pixel intensity quantification across the length of the fiber to demonstrate the continuous gradient.

In some aspects, the mixer 12 can comprise a mixing section 23, and the nozzle 20 can be coupled to the mixing section. Optionally, the nozzle 20 and mixing section 23 of the mixer 12 can be provided as a unitary, monolithic component. In further aspects, the nozzle 20 and mixing section 23 of the mixer 12 can be separate components that are coupled together (and, in exemplary aspects, capable of being selectively decoupled from one another).

The mixer 12 can be configured to couple to the connector 70 via a union such as, for example, a LUER-LOCK fitting. For example, the connector 70 can comprise a female fitting that is configured to receive a male fitting of the mixer 12. The connector 70 can comprise respective channels that extend between and provide fluid communication between the material containers (optionally syringes on syringe pumps or bioprinters) and the mixer 12.

The mixer 12 can define a flow channel 16 that extends along the longitudinal axis 14. The mixer 12 can comprise at least one inlet 18 configured to receive a first printable biomaterial 30 and a second printable biomaterial 32. In exemplary aspects, the mixer 12 can comprise a respective inlet that is configured to receive each printable biomaterial. For example, the mixer 12 can comprise a first inlet 18a and a second inlet 18b. In further aspects, the mixer 12 can comprise a single inlet 18 that is configured to receive each of the printable biomaterials. In some optional aspects, the multicompartmental bioprinting system 10 can be configured to receive at least two materials. For example, the inlet(s) 18 can simultaneously receive two, three, four, five or more printable materials. Optionally, in these aspects, it is contemplated that the printer can simultaneously print two or more materials. In further aspects, the printer can enable the selection of only a subset of the specific materials among the various materials that are to be printed (optionally, simultaneous printed) during a given procedure. For example, when three materials are provided, it is contemplated that the printer 10 can permit printing of a single one of the materials, simultaneous printing of two of the materials, or simultaneous printing of all three materials. In exemplary aspects, the rate of the extrusion of each material can be individually controlled.

One or both of the first and second printable materials (or all of the printable materials in embodiments comprising three or more printable materials) can be biomaterials. At least one of the first material and the second printable material can be a hydrogel. Optionally, at least one of the first material or the second material can comprise a cross-linking agent. Optionally, one of the first material or the second material can be a sacrificial material. For example, one material can comprise gelatin that can be removed to leave behind compartments (e.g., capillaries) as further disclosed herein. Sacrificial materials can be removed during or after the printing process through thermal incubation, diffusion, aspiration, dissolving, or other means. In some exemplary aspects, one or both of the first and second printable materials can comprise photocrosslinkable hydrogels, such as gelatin methacryloyl (GelMA). In further aspects, the biomaterials (e.g., the first printable material, the second printable material, and any additional printable materials) can comprise one or more of a polymer, a protein, nucleic acids, lipids, ceramic, mixture, or other biomaterials. Optionally, in these aspects, the printable material can comprise any polymer such as, for example and without limitation, polycaprolactone, polylactic acid, poly glucolic acid and their co-polymers, alginate, chitosan, etc., or one or more proteins (e.g., collagen or gelatin, or a combination thereof), or combinations thereof. The polymer(s), protein(s) or their mixtures can be functionalized with different chemicals and chemistries. The polymer(s) or their mixture or protein(s) and their mixtures can be mixed with nanoparticles of any shape or microparticles of any shape made or chemicals made from any material composition. Examples include metal (silver, gold, magnesium, zinc, selenium, etc.), metal oxides, metal peroxides, bioglasses, radiopaque agents, antibacterial compounds and agents, antimicrobial compounds and agents, antibiotics, bioceramics, ceramics, oxygen generating materials, crosslinking agents, proteins, vitamins, lipids, phospholipids, fatty acids, biological factors, polysaccharides, nucleic acids, growth factors, hydroxyapetite, calcium phosphate, carbon nanotubes, quaternary ammonium compounds, graphene, graphene oxide, carbon derived materials, liquid crystals, peptides, chitosan, silver nitride, platelet rich plasma, bone marrow-derived materials, pain killers, anti-inflammatory drugs or reagents, blood-derived materials and their combinations, etc. The concentrations of the nanoparticles or microparticles or chemicals can be of any suitable range. Each material can comprise different components such as cells, particles, biological compounds, and/or chemical reagents. The materials can interact with each other upon interfacing, or be affected by external stimuli upon printing, inducing a chemical or physical change in the filament. For example, in various aspects, the biomaterials can crosslink, form secondary structures, form foam, form a gas, or cause destructive or constructive interference. In some aspects, the printed materials can form a filament having two or more different cell types, or growth factors mixed or disposed in distinct regions of the filament.

In some exemplary aspects, the outlet 21 can be spaced from the inlet(s) 18 along the longitudinal axis 16. Optionally, the outlet 20 can be circular and can have a diameter. The mixer 12 can optionally taper in a direction toward the outlet 20.

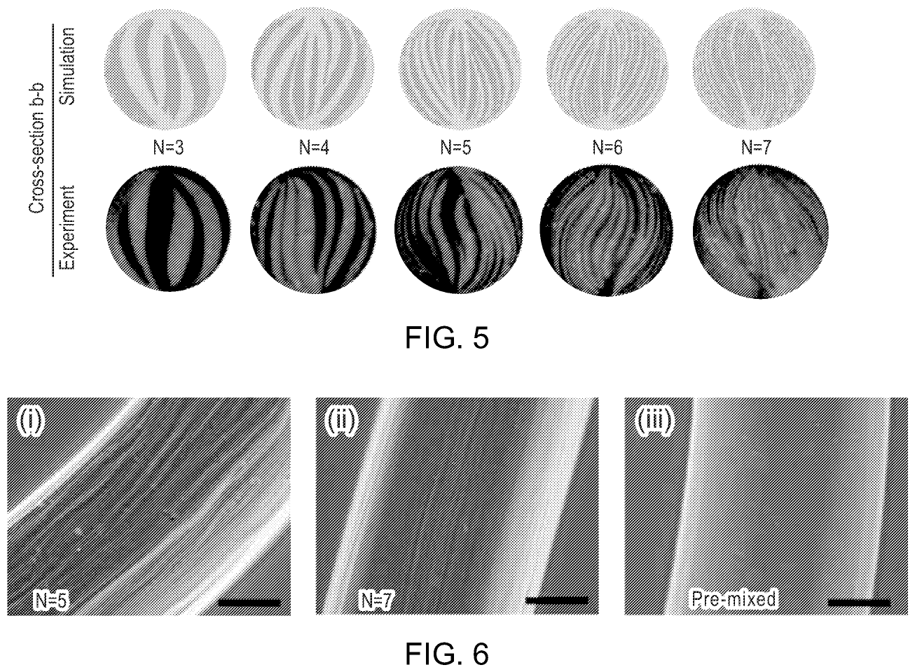

At least one mixing element 22 (e.g., at least one helical mixing element) can be positioned within the flow channel 14 between the inlet 18 and the outlet 20. The mixing element(s) 22 can divide the flow channel into opposed flow channel segments 24. Accordingly, a mixture of the first and second printable biomaterials 30, 32 can be divided into the opposed flow channel segments 24 and then can recombine as further disclosed herein to form a striated mixture, illustrated in FIGS. 5 and 17.

The mixer 12 can be a custom mixer that is configured for use with the particular first and second printable biomaterials 30, 32. For example, it is contemplated that the first and second printable biomaterials 30, 32 can have widely varying viscosities. Accordingly, the mixer 12 can be tailored to form a desired mix profile. Fluid modeling can be used to tailor the mixer 12 (FIG. 2). In some exemplary aspects, to tailor the mixer for a particular set of printable biomaterials, the helical mixing elements 22 can be varied in pitch or length, or position relative to each other. Once the mixer 12 has been designed, in some aspects, the mixer can be formed via additive manufacturing (e.g., 3D printing).

In some aspects, the mixer 12 can couple with a nozzle 20 and coaxial fluidic channels 40. The coaxial channels can have desired length and cross sectional geometries. In some exemplary aspects, to control the size of the fiber 11 and internal organization of the compartments, the size of the coaxial channels can be varied.

The system 10 can comprise at least one actuator 34 that is configured to effect flow of each printable biomaterial. For example, the system 10 can comprise a respective actuator 34 that is configured to control flow of each printable biomaterial. In this way, the flow of the first and second biomaterials 30, 32, or more biomaterials, can be controlled independently. It is contemplated that the respective flow rates of the first and second printable biomaterials 30, 32 can be selected based on the material properties (e.g., viscosity) of the first and second printable biomaterials, printing rate, and fiber 11 dimensions. Each actuator 34 can be, for example, a pump, hydraulic cylinder, an automatic system such as a commercial (bio)printer, or any suitable device for effecting controlled flow of the printable biomaterials.

The system 10 can comprise an automatic position controller to control the position of the depositing fibers. The automatic position controlled can be a stationary robot or a robotic arm. The position of the fiber deposition can also be controlled manually by moving the system 10 using hand movement.

The system 10 can comprise a sleeve 40 that surrounds the outlet 21 of the nozzle 20 to define a coaxial fluidic system. The sleeve 40 can comprise an inlet 42 that is configured to receive an additional biomaterial and/or a crosslinking agent (collectively, sheath material 44). The crosslinker can be any suitable crosslinker such as ionic, chemical, and enzymatic crosslinker. For example, in some aspects, the crosslinker can be CaCl₂ crosslinking alginate, one of the extruded biomaterials. Another actuator 46 can be configured to effect flow of the sheath material 44, e.g., from an additional biomaterial and/or a crosslinking agent supply 47. A computing device 1001, further disclosed herein, can be configured to control all actuators 34, 46 to thereby form fibers 11 having desired structures.

Referring also to FIG. 7B, the sleeve 40 can comprise an outlet 48. In some optional aspects the outlet 48 of the sleeve 30 can be is spaced from the outlet 20 of the mixer 12 along the longitudinal axis 14 of the mixer in a direction away from the at least one inlet 18 (FIG. 3) of the mixer.

In exemplary aspects, the mixer 12 can be coupled to a robotic arm for controlled delivery of the fiber 11. The robotic arm can be a 3-axis arm. In further aspects, the robotic arm can be a four, five, or six axis robotic arm. In still further aspects, the mixer 12 can be handheld so that a clinician can manually control deposition of the fiber 11. In yet further aspects, the mixer 12 can be held in a fixed position.

Referring to FIG. 17,18, in some aspects, the first printable biomaterial 30 can be a sacrificial biomaterial. In further aspects, the second printable biomaterial 32 is a hydrogel. In this way, the sacrificial biomaterial can be removed from the fiber 11 to leave flow channels 60. The flow channels 60 can have axes that are parallel to, or generally parallel to, the central axis of the fiber 11, or have a different angle, as demonstrated by compartments with a desired angle with respect to the fiber 11 in FIG. 12.

The sheath material can be flowed through the sleeve 40 that surrounds the outlet 21 of the nozzle 20. In some aspects, and referring also to FIG. 7, the flow rate of the sheath material can be controlled relative to a cumulative flow rate of the first and second (and other) printable biomaterials. In this way, the diameter of the fiber can be controlled. For example, the outlet of the nozzle can have a diameter. The flow rate of the sheath material relative to the cumulative flow rate of the first and second printable biomaterials can be selected to form a fiber having a diameter that is more or less than the diameter of the outlet 21 of the nozzle 20. As further described herein, it is contemplated that narrowing the outlet 21 of the nozzle 20 can cause excessive shear that kills cells. By selecting the diameter of the fiber by flow of the sheath material 44, the shear can be reduced.

In some aspects and with reference to FIGS. 8 and 12, extruding the first and second (and, optionally, additional) printable biomaterials 30, 32 through the mixer 12 can form longitudinally extending striations. The flow rate of the sheath material relative to the cumulative flow rate of the first and second (and other) printable biomaterials can be selected to deviate at least a portion of the longitudinally extending striations to have a controlled angle relative to fiber axis 11. For example, a greater flow rate of the sheath material relative to the cumulative flow rate of the first and second printable biomaterials can cause greater deviation in the radial direction, and a lesser flow rate of the sheath material can form lesser deviation in the radial direction. The deviation can be radially outward deviation from parallel to a central axis of the fiber. Accordingly, the striations can intersect an outer perimeter of adjoining portions the fiber at an angle, wherein a greater flow rate of the sheath material 44 can cause the striations to intersect the outer perimeter of the fiber at an angle closer to 90 degrees, and a lower flow rate of the sheath material 44 can cause the striations to intersect the outer perimeter of the fiber at an angle closer to zero degrees. In exemplary aspects, the relative flows of the printable biomaterials and the sheath material 44 can be selected so that at least a portion of the striations (optionally, the entirety of the striations) intersect the outer perimeter at an angle from 30 degrees and 90 degrees, or from about 45 degrees to about 90 degrees, or from about 60 degrees to about 90 degrees, or from about 45 degrees to about 60 degrees.

In some aspects, at least one of the biomaterials forming the fiber 11 can be crosslinked through various crosslinking mechanisms including irradiation (e.g., ultraviolet radiation), ionic crosslinking, chemical crosslinking, and/or enzymatic crosslinking.

Referring to FIGS. 14-16, in some aspects, at least a bioprinting method or one biotextile technique can be applied to the fiber 11 with at least one additional fiber to form a biomimetic assembly 64 of multicompartmental fibers. In some optional aspects, the at least one biotextile technique can comprises (i) weaving, (ii) braiding, (iii) knotting, (iv) coil formation, or a combination thereof.

### Method of Use

At least first and second biomaterials can be extruded from at least the first and second containers. The mixer can mix the at least first and second biomaterials to form a mixture having a predetermined cross sectional structure. The mixture can be deposited through the nozzle to form a filament.

After the deposition of the filament, the filament can be crosslinked. The crosslinking can be performed via known physical, thermal, chemical, ionic, and/or enzymatic methods, or combinations thereof. In further aspects, crosslinking can be provided by external sources or internal material interactions. For example, a first biomaterial can act as a source of ions to crosslink a second biomaterial through diffusion. The printer can control the degree of crosslinking in the interaction with precursor materials. Further, the printer can use sacrificial (noncrosslinked materials) that can be removed to generate unique architectures and features in the extruded filaments. This can be used to generate vasculature and topographical cues for certain biological applications. For example, gelatin can be printed as circular coaxial compartments within a GelMA outer structure whereby the gelatin can be sacrificed after the GelMA outer structure is crosslinked. In this case, gelatin can melt and/or dissolve (or be otherwise removed) to create lumens (e.g., microlumens).

Optionally, the outlet 21 of the nozzle 20 can be positioned in a support bath or other environmental conditions like vapor to aid physical support, structural complexity, crosslinking, or some other feature of the extrusion process.

### EXAMPLE EMBODIMENTS

The multicompartmental bioprinting system 10 can be configured to mix the precursor materials to provide a particular ratio, gradient, or cross sectional structure and form a multicompartmental fiber or droplet. In this way, the system 10 can be used to construct or reconstruct a desired architecture or scaffold, for engineering of tissue in regenerative medicine, in vitro tissue modeling and food biomanufacturing.

In some optional aspects, the printer can be coupled to a robotic arm or a manual, surgeon-guided mechanical device (e.g., a mechanical arm that moves in response to manual actuation and/or control). It is contemplated that the robotic arm can enhance the resolution or accessibility to the wound of the printing.

The developed multicompartmental bioprinting system 10 can address the requirements for tissue biofabrication. Particularly, the following (main) requirements were tested: (A) formation of high cell-scale features within each filament for guiding cellular organization; (B) formation of capillaries (e.g., microcapillaries) to improve nutrition transfer and help vascularization of the scaffold; (C) formation of a gradient across a single fiber for printing scaffolds at the interface of two different tissues.

The multicompartmental bioprinting system 10 can be configured to print multicompartmental hydrogel fibers for skeletal muscle tissue engineering. Generally, there are two important challenges that can affect the formation of functional and aligned contractile muscle fibers: 1) lack of topographical cues in hydrogel fiber with dimeters larger than 500 μm; and 2) diffusion limit of nutrients and oxygen in such fibers. The survival of implanted cell-laden scaffolds can be dependent on oxygenation by its connection to the blood circulation of the host body. The physiological process of angiogenesis within thick and large sized implants is time-consuming, which results in the failure of clinically sized implants due to massive starvation-induced cell death within the implant. Hollow microchannel incorporated three-dimensional (3D) scaffolds have shown faster angiogenesis in vivo due to the microchannels, which induced the rapid recruitment of satellite cells into the implants. Embodiments disclosed herein can overcome these challenges.

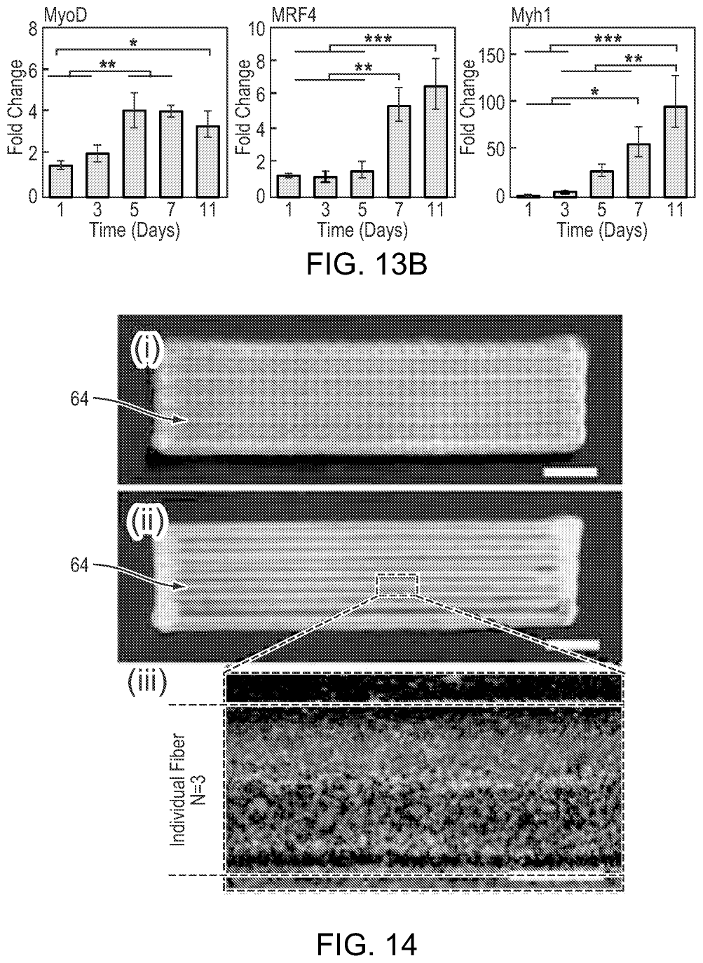

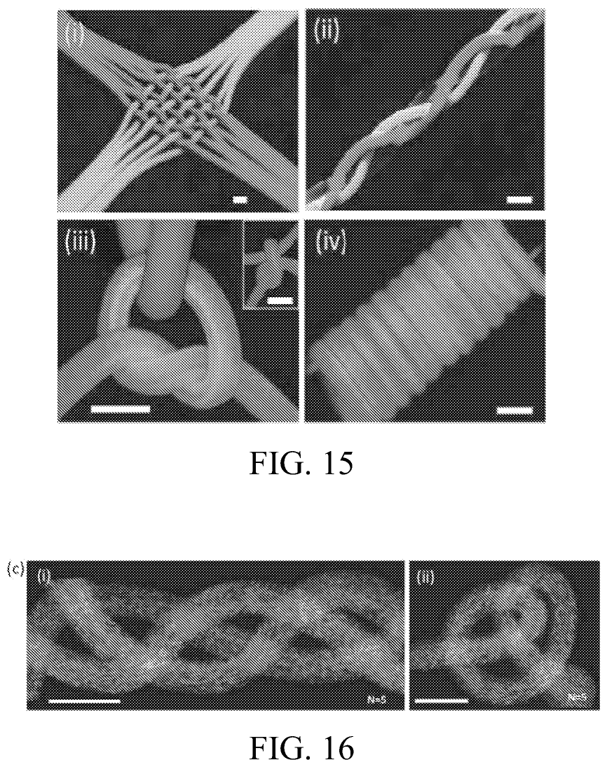

Controlling cellular organization is crucial in the biofabrication of tissue-engineered scaffolds, as it affects cell behavior as well as the functionality of mature tissue. Thus far, incorporation of physiochemical cues with cell-size resolution in three-dimensional (3D) scaffolds has proven to be a challenging strategy to direct the desired cellular organization. Using the multicompartmental bioprinting system 10 disclosed herein, a rapid, simple, and cost-effective approach is developed for continuous printing of multicompartmental hydrogel fibers with intrinsic 3D microfilaments to control cellular orientation. A static mixer 12 integrated into a coaxial microfluidic device 40 can be utilized to co-print alginate 30 and gelatin-methacryloyl (GelMA) 32 hydrogel fibers 11 (e.g., meter-long fibers) that embed sequential filaments of alginate and GelMA. In the engineered microstructure, GelMA compartments provide a cell-favorable environment, while alginate compartments offer morphological and mechanical cues that direct the cellular orientation. Numerical simulations (FIG. 2) show that, while the resolution (size of internal compartments) can be decreased down to cell-size dimensions, the shear stress is not increased and therefore the behavior of the cells are not affected. The thickness of the fiber can be controlled by changing the core-to-sheath flowrate ratio. The size of internal filaments can be manipulated from millimeter to micrometer scales through changing the number of helical mixing elements (FIG. 5). It is further demonstrated that the organization of the microtopographies, and, consequently, the cellular alignment can be tailored by controlling flow parameters in the printing process (FIG. 12). Despite the large diameter of the fibers, the precisely tuned internal microtopographies can induce excellent cell spreading and alignment (FIGS. 9 and 10), which facilitate rapid cell proliferation (FIG. 11) and differentiation (FIG. 13) toward mature biofabricated constructs. This strategy can advance the engineering of functional tissues. The multicompartmental bioprinting system 10 can further be integrated with any bioprinting tool or fiber spinning strategy. The printed multicompartmental fibers can be assembled through bioprinting (FIG. 14) or biotextile approaches (FIGS. 15 and 16) to form higher scale 3D complex tissue constructs.

### Example 1

The process of fabricating multicompartmental hydrogel fibers (MCHFs) is depicted in FIG. 1. The disclosed systems and methods are based on the manipulation of different hydrogels' flow for construction of a compartmentalized stream of the bioink. Alginate and GelMA were selected as hydrogels for this purpose. One of the main challenges in fiber-based biofabrication approaches, such as extrusion bioprinting and biotextile manufacturing, is the selection of a “cell-favorable” bioink that can form a scaffold with high shape fidelity. This can use a relatively viscous precursor that can rapidly crosslink upon printing to form a robust and stable fiber.

GelMA can be a cell-permissive hydrogel that supports cell spreading and proliferation due to presence of cell attachment sites such as arginine-glycine-aspartic acid (RGD) peptides, as well as matrix metalloproteinase (MMP) sensitive degradation motifs, suitable for cell remodeling. However, due to its low viscosity and non-instantaneous photocrosslinking, direct formation of stable GelMA fibers can be challenging. A possible solution to overcome this is the incorporation of other hydrogels to enable GelMA fiber formation. Alginate is a good candidate for mixing because it exhibits the necessary viscosity and rapid ionic gelation. A hybrid GelMA/alginate bioink can easily be implemented in bioprinting or biotextile strategies.

Incorporation of alginate, which lacks cell attachment sites and biodegradable peptides, can reduce the suitability of such bioinks for tissue engineering applications. As disclosed herein, the lack of cell attachment sites in alginate can be addressed, through compartmentalization of the bioinks in such a way that distinct GelMA compartments support cell functionality while alginate compartments enable quick formation of stable fibers. A static mixer integrated coaxial microfluidic device can be employed for fabrication of MCHFs. A static mixer with an optimized number of mixing elements can be implemented to divide the main streams of alginate and GelMA solutions into sub-streams with the desired thickness. This solution, with intercalated striations of GelMA and alginate, can then be extruded through the inner channel of a coaxial microfluidic device and exposed to Ca2+ ions to stabilize the structure through gelation of the alginate. At this stage, the crosslinked alginate can physically confine the striations of GelMA precursor. UV irradiation can subsequently be used to crosslink the GelMA within the alginate matrix and form internal microfilaments. The final structure can be a millimeter-scale hydrogel fiber with micro-scale internal topological features, consisting of consecutive microfilaments of alginate and GelMA hydrogel. This multiscale fibrous structure can enable cells' spreading and alignment.

The subdivision of the different hydrogel streams into micro-scale sub-streams, embedded within the millimeter-scale flow, can lead to formation of internal features of a much smaller size than the diameter of the printed filament. In conventional bioprinters, the minimum feature size is dictated by the nozzle diameter. As a result, improving the resolution comes at the cost of an increase in the shear stress applied to the encapsulated cells as well as an elevated pressure required for extrusion of viscous bioinks through the smaller nozzle. However, in the disclosed printing strategy, resolutions is not shear-dependent and can be improved through the consecutive sub-division of streams without changing the nozzle diameter. Numerical simulation results demonstrate that while increasing the resolution using the static mixer does not significantly increase the shear stress inside the flow, a corresponding decrease in nozzle tip diameter to match the resolution can be enhanced with each additional static mixer element can increase the shear stress by ˜8 fold. Similarly, the extrusion pressure is not significantly increased by the static mixer due to its relatively large channel size (e.g., ˜5 mm), while the pressure increased by ˜15 fold with decreasing the size of nozzle tip corresponding to the application of each additional static mixer element.

Alignment of cells within the hydrogel fiber, further disclosed herein, can establish a hierarchical multiscale construct, mimicking the structure of native fibrillar tissue. The fabrication method disclosed herein is simple and cost-effective, without any requirement for special tools. In addition, its high throughput allows the fabrication of cell-laden fibers at speeds up to meters per minute and makes this method attractive for unconventional applications of tissue engineering that requires mass production, such as lab-grown meat.

FIG. 1 illustrates Biofabrication of multicompartmental hydrogel fibers for formation of multiscale biomimetic constructs. FIG. 1 shows an exemplary fabrication setup comprising a static mixer creating striations of different hydrogels, integrated with a coaxial microfluidic device extruding the mixed streams of hydrogels through a sheath flow of CaCl₂ to crosslink alginate and form the matrix of the fiber. The fibers can then be exposed to UV light to crosslink the GelMA striations within the alginate matrix, creating an internal fibrous microstructure. A millimeter-scale filament with micro-scale internal filaments can be formed using the disclosed bioprinting method. Referring to FIG. 2, fluid dynamics simulations can demonstrate that increasing the resolution of the multimaterial bioprinting using the static mixer does not significantly increase the shear stress and required extrusion pressure. The shear stress (t) is map in the static mixer (i) and conical nozzles (ii) with different tip diameters. The tip diameter (D) was reduced by half in different simulations, corresponding to the application of each static mixer element subdividing the upper stream into two half-sized sub-streams. The shear stress across the nozzle tips relative to the maximum shear stress in the static mixer (τₘₐₓ) is graphed (iii). The pressure (P) map in the (iv) static mixer and (v) conical nozzles with different tip diameters. The pressure inside the conical nozzles relative to the maximum pressure inside the static mixer (Pmax) is graphed (vi). FIG. 9A shows the organized internal microstructure of the fibers directs cellular alignment, while the robustness of the fibers enables their bioassembly, toward formation of biomimetic hierarchical structures. The sizes of scale bars are indicated by dotted red arrows. F-actin/DAPI staining was used here to assess the morphology of C2Cl2 cells cultured in multicompartmental fibers.

Referring to FIGS. 4-5, formation of MCHFs with internal microfilaments can be controlled by controlling mixing of the two constituent precursors in the mixing nozzle. A computational finite element simulation was implemented to elucidate the working principle of the static mixer integrated coaxial microfluidic device. FIG. 2 shows the computer aided design (CAD) model of the static mixer used for the simulations. In the disclosed example, a static mixer, comprising multiple helical elements twisting intermittently in different directions, was used for formation of MCHFs. The static mixer can be based on a KENICS static mixer manufactured by Chemineer of Dayton, OH. As indicated by simulations shown in FIG. 5, each Kenics element in this setup divides the upstream of the flow into two sub-streams. By injecting two different solutions into the mixer, the streams can be consecutively divided into more sub-streams, forming an array of different striations. The total number of striations created using an N-element static mixer is therefore 2N, while the number of striations for each component will be 2N−1. Assuming a uniform distribution, the thickness of each striation is then Df/2N, where Df is the final fiber diameter. Consequently, by controlling the number of elements in the static mixer, an internal structure with tunable thickness and number of striations can be formed. The cross section of flow clearly demonstrates the formed striations within the flow (FIG. 5, top row).

The simulation results were validated experimentally, as shown in the bottom row of FIG. 5. The helical mixing element CAD design was 3D printed using a stereolithography 3D printer, followed by its insertion into a barrel and integration with a coaxial microfluidic device. The device was then used for evaluation of the flow profile generated by the static mixer. Immediate crosslinking of the structure through wet spinning of alginate into a CaCl₂ bath can preserve the internal microstructure of the fabricated fibers for analysis. Examining cross-sections of experimentally generated fibers confirmed the formation of striations within the flow, which were crosslinked and formed the internal microfilaments. FIG. 5, bottom row, indicates the size dependency of the microfilaments to the number of the mixer elements.

Multicompartmental alginate/GelMA fibers were fabricated using the two-step crosslinking process described above. FIG. 6 indicates the effect of mixing level on the internal microstructure of the fibers fabricated using this method. As expected, a fibrous structure can be generated in which increasing the number of Kenics elements decreased the size of internal microfilaments. Comparatively, a pre-mixed bioink, prepared via vortex-mixing and extruded through static mixer-integrated microfluidic co-axial device, formed a homogeneous fiber without internal microfilaments.

To demonstrate that the developed multicompartmental printing is not limited to the implemented materials (alginate and GelMA) or their specific crosslinking methods, the compatibility of the strategy with two different materials including Pluronic-F127 and Laponite nanoclay hydrogels was evaluated. The results demonstrated that the internal microfilaments could be easily formed and preserved upon printing.

The effect of the coaxial microfluidic device on the hydrogel fiber structure was investigated. The primary role of the coaxial microchannels is the induction of alginate gelation, making the fabrication strategy compatible with extrusion-based bioprinting. The coaxial system further provides the opportunity of accurate control over the diameter of fabricated hydrogel fibers, as illustrated in FIGS. 7A and 7B. Although the diameter of the fabricated fiber can also be adjusted by changing the size of the nozzle outlet, tuning the ratio of outer (CaCl2 solution) channel flow rate, Qout, to that of inner (multicompartmental hydrogel solution) channel, Qin, offers a real-time and accurate control over the size of final fiber. Simulation and experimental results demonstrated that by adjusting the Qout/Qin ratio, the orientation of internal microfilaments can be manipulated. While a ratio of Qout/Qin≈1-2 did not significantly change the orientation of formed internal microfilaments, a higher ratio could deform the streamlines, as shown by simulation results. Immediate gelation of alginate upon exposure to Ca2+ ions could preserve the formed microstructure, and even intensify it by solidifying the outer layers of the fiber while the fluid is still flowing in the inner layers.

The capability to independently tune both the size of the final fiber and its internal microfilaments provides the opportunity to implement current extrusion-based bioprinters while improving resolution down to cell-size scales. This multiscale biofabrication strategy specifically offers the formation of fibrous tissues with any target size while maintaining the capacity of the scaffold to direct cellular organization. The multicompartmental microstructure further provides the opportunity to harness the advantages of different biomaterials.

FIGS. 6-8 illustrate characterization of multicompartmental hydrogel fibers. FIG. 3 show a representative design of a static mixer with helical elements used for flow characterization via finite element simulations. FIG. 4 shows a fluid model showing the working principle of the static mixer demonstrated using simulation results. Two streams of hydrogel precursors were introduced at the inlets of the static mixer and then consecutively divided into sub-streams by Kenics elements, followed by their blending as a result of helical profile of the elements. FIG. 5 shows a cross-section of the multicompartmental stream (top row, simulation results) or fabricated hydrogel fiber (bottom row, experimental results), demonstrating the effect of number of elements on the number and size of internal microstructure. N stands for the number of consecutive elements in the mixer. Scale bar is 500 μm. FIG. 6 shows phase contrast images of multicompartmental alginate/GelMA fibers demonstrating the fiber's internal microfilaments. Increasing the number of mixing elements decreased the size of the microfilaments. Subfigures (i) and (ii) correspond to the fibers fabricated using the Kenics static mixer with 5 and 7 elements, respectively, while (iii) shows the fabricated fiber with a pre-mixed bioink, prepared through vortex-mixing and heating at 80° C. Scale bars are 200 μm. FIG. 7A is a plot showing the control over the fiber diameter using the coaxial microfluidic device. While the diameter of the fiber can be manipulated by changing the diameter of the internal channel in coaxial device, it can also be tuned finely by the adjusting the inner- and outer-channel flow rates (Qᵢₙ and Qₒᵤₜ, respectively). Radius of the fiber (R_{fiber}) and Radius of the inner channel (R_{inner channel}) indicate the radius of the fabricated hydrogel fiber and radius of inner channel in coaxial microfluidic device, respectively. n=3 for each measurement point. FIG. 7B shows simulation results demonstrating the effect of Qₒᵤₜ/Qᵢₙ ratio on the diameter of the fabricated fiber. Blue streamlines show the flow of CaCl₂ and yellow streamlines represent the hydrogel mixture flow (Qₒᵤₜ/Qᵢₙ=1, 3 and 8, respectively from left to right). FIG. 8 shows a portion of a simulation and a fiber formed in accordance with the simulation, showing the effect of flow rates on organization of internal microfilaments. Increasing the Qₒᵤₜ/Qᵢₙ ratio deforms the streamlines of the hydrogel mixture and therefore changes the orientation of the internal microfilaments. Scale bar is 200 μm.

FIGS. 9A, 9B, and 12A illustrate cellular organization and metabolic activity in multicompartmental hydrogel fibers. FIG. 9A shows F-Actin/DAPI staining demonstrating the alignment of myoblasts along the fiber axis 24 h post-encapsulation. A static mixer with six Kenics elements was implemented for fabrication of multicompartmental alginate/GelMA fibers. The bottom image is a magnified representation of the zone indicated by dashed rectangle in the top image. In contrast to the cells cultured in multicompartmental fibers, FIG. 9B illustrates F-Actin/DAPI staining of myoblasts of those cultured in pre-mixed hybrid hydrogel fibers that didn't demonstrate spreading or alignment. The bottom image is a magnified representation of the zone indicated by dashed rectangle in the top image. FIG. 10 shows plots of quantitative evaluation of F-actin cytoskeleton (left) and nuclei (right) directionality within MCHF compared with hydrogel fibers fabricated from pre-mixed bioink. Although the size of the fibers was large compared to cells' dimension (˜50 times), a highly aligned unidirectional organization was observed both in the cytoskeleton and nuclei of the cells cultured in the MCHFs (θ is shown in A). FIG. 11 is a plot showing enhanced metabolic activity of the cells cultured in MCHFs compared to the cells cultured in pre-mixed fibers. n=4 for each time point. Scale bars are 500 μm for the top row and 200 μm for the bottom magnified images.

The multicompartmental fiber biofabrication strategy enabled directing cellular organization. Cells were encapsulated in GelMA precursor and MCHFs were fabricated as previously described. FIGS. 9A-9B illustrate a comparison of the behavior of myoblasts cultured within the MCHFs with those cultured in fibers fabricated with pre-mixed bioink. Despite the large (>1 mm) diameter of the fibers compared to the cell size, a highly aligned cellular organization was observed in MCHFs 24 h post-fabrication, while the cells encapsulated in pre-mixed fibers remained almost spherical. The cellular alignment within the multicompartmental hydrogel fibers can be explained by (i) differential favorability of the cells for spreading in GelMA microfilaments over the alginate sections, (ii) fibrous internal microstructure acting as topological cues for directing cellular alignment; and (iii) mechanical stimulation of the cells due to differential mapping of scaffold stiffness in GelMA and alginate sections.

Since alginate does not have bioactive sequences, it acts as a cell repellant compartment in the fiber structure, and therefore induces cell spreading inside internal GelMA microfilaments. Furthermore, the presence of 3D microtopographies of comparable size to the cell dimensions can direct cellular alignment along the microcompartments interfaces. As described previously, for a fiber with a diameter of approximately 1 mm, a static mixer with 5-6 Kenics elements forms internal GelMA microfilaments with an average size of 15-30 μm. The data indicates that higher level of mixing leads to formation of fibers without distinct regions due to miscibility of aqueous GelMA and alginate precursors. Upon exposure to Ca²⁺ ions, alginate immediately crosslinks, which is accompanied by structure shrinkage, squeezing out the liquid GelMA from the construct before photocrosslinking. A decreased level of mixing, which consequently reduces the entrapment of the GelMA striations, therefore causes leaching of large portions of GelMA, leaving behind only non-cell-permissive alginate.

The difference in mechanical properties of alginate and GelMA hydrogels can further induce cellular alignment as a result of mechanical stimulation. FIG. 13A demonstrates the significant difference between mechanical properties of the alginate and GelMA hydrogels used in this study. It has been shown that the presence of stiff geometrical constraints (anchoring sites), which can restrict the movement of cell-containing hydrogels, induce cellular alignment and maturation, specifically in contractile tissues. The cellular alignment in these systems arises from mechanical stimulation generated by a cytoskeleton mediated internal tension along the lines passing between the hydrogel anchoring sites. Many studies have reported the application of stiff geometrical constraints for anchoring the cell-laden hydrogel and therefore inducing cellular alignment. Specifically, it has been demonstrated that an alignment in the geometry of stiff anchoring sites can align cells more effectively. In the disclosed system, aligned alginate microfilaments with significantly higher elastic modulus compared to GelMA can act as anchoring sites, constraining the cell-laden GelMA hydrogel, and therefore induce alignment. The application of alginate as a stiff hydrogel within soft hydrogel networks has been previously reported for controlling cellular shape and spreading.

FIGS. 7A-7B, 12A, and 12B illustrate real-time control of cellular organization within the multicompartmental fibers. FIG. 12A shows the effect of Qₒᵤₜ/Qᵢₙ ratio on cellular alignment. Increasing the ratio can deviate the orientation of the cells in the fibers by deforming the hydrogel flow striations, and therefore internal microfilaments direction. Upper subfigures show the results of fluid dynamics simulations at the outlet of the microfluidic coaxial channels (Qₒᵤₜ/Qᵢₙ=3, 8 and 10, respectively from left to right) while lower subfigures show the corresponding cellular arrangement, demonstrated using F-actin/DAPI staining. Dashed-dotted lines indicate centerlines. Scale bars are 500 μm. FIG. 12B shows a distribution of F-actin orientation at different ratio of flow rates, corresponding to the images shown in FIG. 12A. A static mixer with five Kenics elements was used for formation of internal microfilaments.

A quantitative evaluation of cell orientation in the multicompartmental hydrogel fibers demonstrated an almost uniaxial organization of both cytoskeleton and nuclei along the fiber axis. The alignment of the nuclei is of specific importance due to the crucial role of nuclei morphology in cellular behavior, affecting their metabolic activity, protein expression and differentiation. It was further demonstrated that the multicompartmental hydrogel fibers support cellular proliferation, in contrast to the fibers fabricated from the pre-mixed bioink. The presence of distinct GelMA regions in the engineered construct ensures cell spreading and proliferation. However, in the pre-mixed structure, the presence of alginate does not allow scaffold degradation and therefore does not offer enough space for proper cell spreading and proliferation. As a result, the activity of the cells, and therefore their rate of proliferation, decreased over time.

The potential of the proposed biofabrication strategy for directing alignment of the cells along the fiber axis has been demonstrated, while supporting cellular activity and function. Since cells follow the fiber direction, their orientation inside the scaffold can be easily controlled by adjusting the orientation of the fiber during bioprinting or assembly of the fibers through biotextile methods. The ability to control cellular alignment inside the individual fibers has further been demonstrated. Manipulation of flow rates in the microfluidic coaxial device can provide the opportunity to change the orientation of internal hydrogel microfilaments. This advantage can be exploited to control the internal organization of the cells. Because the encapsulated cells spread along the internal microfilaments, the cellular alignment can be finely tuned by controlling the flow rates. As shown in FIG. 12A, increasing the ratio of Qₒᵤₜ/Qᵢₙ can deviate the direction of cellular orientation from the fiber axis toward a radial alignment, perpendicular to the fiber axis. While a static mixer with both 5 and 6 mixer elements could effectively generate MCHFs with controlled cellular organization, five mixer elements were used here to generate larger features and better detect and characterize the cellular directionality. The quantitative evaluation of F-Actin direction indicates a unidirectional orientation in the angled arrangement. The adjustment of cellular orientation with flow rates enables a continuous real-time control over the cellular organization within the final scaffold.

FIGS. 13A-B show the application of multicompartmental hydrogel fibers as a scaffold for muscle tissue engineering. FIG. 12A shows a morphology analysis of encapsulated myoblasts over a week using F-Actin/DAPI staining. As illustrated, highly aligned cells rapidly proliferated, fused with each other, and differentiated toward muscle fiber formation. The bottom row represents the magnified images of the zones indicated in top row by dashed rectangles. Scale bars are 200 μm for the top row and 100 μm for the bottom row. FIG. 13B shows plots indicating myogenic progression of the cells in hydrogel fibers using gene expression analysis with RT-qPCR. The expression of early (MyoD) and late (MRF4 and Myh1) myogenic markers was evaluated over 11 days. Fold change is calculated by normalizing the results to GAPDH as internal reference and Day 0 results. n=3 for each time point.

Fiber based biofabrication approaches can be employed in production of biomimetic scaffolds for engineering anisotropic tissues such as muscle. Mimicking fibrillar structure of such tissues in bioengineered scaffolds can regulate encapsulated cells' behavior toward enhanced myogenesis. Referring to FIGS. 13A-B, the ability of the proposed strategy for supporting myoblast maturation has been demonstrated. The fibers were fabricated using the previously described strategy, with a static mixer having six mixing elements. A Qₒᵤₜ/Qᵢₙ=1 ratio was applied in the coaxial microfluidic system to ensure the alignment of the microfilaments and therefore the encapsulated cells along the fiber axis. Following fiber fabrication and their subsequent culture for 24 h to allow cellular alignment, the maturation of the myoblast was investigated by evaluating the morphology and gene expression of the cells over time. As indicated in FIG. 19, the aligned myoblasts rapidly proliferated, fused, and formed multinucleated myotubes. On day seven post-encapsulation, the hydrogel fiber was completely occupied by highly-oriented densely-packed myotubes, forming a fascicle-like structure.

To confirm the results obtained from the morphology analysis, the expression of myogenic markers from the myoblast-laden multicompartmental hydrogel fibers was evaluated. The transcriptional level of early and late myogenic markers was examined over time using RT-qPCR. In muscle tissue formation, myogenic regulatory factors (MRFs) including Myogenic Differentiation (MyoD), and MRF4 can control the differentiation of cells toward myofibers. The myogenic progression of encapsulated myoblast cells can be shown. At the initial differentiation step, aligned myoblasts form myocytes and fuse with each other. These cells then experience secondary fusion, creating myotubes, which can further form muscle fibers. Finally, these myofibers mature to form fascicle-like constructs. In this process, MyoD induce the expression of Myogenin which is necessary for myocyte formation and fusion. In addition, MRF4 plays a dual role, active both in proliferation of undifferentiated myoblasts and as a differentiation gene in cells undergoing maturation. It has been also reported that both Myogenin and MRF4 contribute to terminal differentiation. Finally, in the matured muscle, sarcomere contractile proteins such as Myosin Heavy Chain 1 (Myh1) are highly expressed while MRF4 transcript levels exceed the expression of other MRFs. In the disclosed example, the expression of MyoD in the cell-laden multicompartmental fibers peaked in days 5-7, indicating the differentiation of myoblasts, while the MRF4 level showed a sharp increase on day 7, demonstrating the maturation of the differentiated cells. High levels of myosin heavy chain expression on day 11 further confirm the maturation of cells and formation of fascicle-like structure.

FIGS. 14-16 illustrate application of multicompartmental hydrogel fibers for biofabrication of higher-scale constructs. FIG. 14 shows bioprinting of multi-layered mesh (i) and unidirectional structures (ii). The microscopic picture (iii) confirms preservation of the internal microfilaments generated by a static mixer upon printing. Scale bars are 5 mm for (i) and (ii), and 500 μm for (iii). A mixer with three mixing elements was used for better visibility of different compartments along the fibers. FIG. 15 shows various biotextile techniques including (i) weaving, (ii) braiding, (iii) knotting, and (iv) coil formation, for biomimetic assembly of multicompartmental hydrogel fibers. The capability for manipulation of the structure of assembled constructs by controlling individual fiber composition is indicated by encapsulation of two different fluorescent particles in the fibers. Scale bars are 2 mm. FIG. 16 shows cell-laden constructs fabricated through bio-textile assembly of multicompartmental hydrogels. Actin-DAPI assay was used for staining. Scale bars are 2 mm.

One of the most important advantages of the proposed hydrogel fiber formation is compatibility with existing fiber-based biofabrication methods for constructing higher scale structures with physiologically relevant dimensions. Using an extrusion bioprinting device as disclosed herein, fibers were deposited to form a multicompartmental two-layer mesh (FIG. 14; (i)) or unidirectional fibrous structures, suitable for mimicking anisotropic tissues (FIG. 14; (ii)). A microscopic picture of the printed structure demonstrates that upon printing, the internal microfilaments formed by the static mixer were preserved (FIG. 14; (iii)). As illustrated in FIG. 15, the fibers can also be fabricated by wet spinning and assembled using various biotextile approaches. The multicompartmental fibers were mechanically strong enough to allow easy handling. Since the proposed fiber fabrication strategy enables production of relatively large fibers, while preserving the required resolution, the handling challenges would be further reduced. In addition, the large size of the fibers offers minimal assembly steps for production of tissue-scale constructs. FIG. 16 shows cell-laden assembled constructs fabricated through biotextile processes. The capability for manipulation of the structure of assembled construct by adjusting the composition, microstructure, and cellular orientation of individual fibers offers a high level of controllability in the biofabrication strategy.

Discussion of Example 1

Controlling cellular organization in biofabrication strategies is one of the most important, but challenging, requirements in engineering of highly organized tissues. This includes biomimetic spatial distribution of the cells as well as specific cellular alignment within the scaffolds. While the spatial distribution of the cells in the scaffolds can be controlled by various top-down or bottom-up biofabrication approaches, controlling the alignment of the cells during biofabrication is still an unmet need. The precise mimicking of cellular organization in biofabrication can not only to regulate encapsulated cells' behavior toward formation of the target tissue, but can also to promote the functionality of the final maturated tissue. For example, the proper alignment of cells within a muscle enhances the force generation capacity of the tissue. To form such cellular organization, a biofabrication strategy enabling high resolution control over the microstructure and patterned biomaterials is disclosed. The resolution in the order of cell dimensions can ensure a proper regulation of cellular alignment within the scaffold. To address this demand, the disclosed methodology was designed based on two key elements:
A robust biofabrication strategy that can form scaffolds with controlled microstructure with feature size in the order of the cell dimension; is compatible with bioprinting and biotextile assembly methods; and is simple, low cost, and high throughput; and
A suitable bioink that supports cell functionality by providing binding sites and biodegradable sequences; forms stiff microtopographies to direct cellular alignment; enables rapid crosslinking for compatibility with bioprinting and fiber spinning approaches; and is mechanically strong enough to form a scaffold with high shape fidelity.

The biofabrication requirements were addressed by development of a method having precise control over the flow of different hydrogel precursors in a microfluidic nozzle. A static mixer with an optimized number of helical elements was used to divide streams of different hydrogel precursor and create aligned striations of hydrogels with desired dimensions, comparable to the cell size. Computational fluid dynamics simulations demonstrate that the resolution in this system is not shear- or pressure-dependent and is improved through the consecutive sub-division of streams without changing the nozzle diameter. This is an important advantage in fluidic systems, specifically microfluidic devices applied in biofabrication of cell-laden constructs. Conventionally, the resolution in extrusion bioprinting is increased with the nozzle diameter. A fine nozzle diameter increases the shear stress applied to the encapsulated cells and significantly decreases their viability. Additionally, the high pressure drop in fine nozzles, due to energy dissipation by channel wall-mediated hydrodynamic resistance against the fluid flow, necessitates higher extrusion pressure. A higher extrusion pressure can affect the cellular viability and requires the application of pumps with higher power as well as better channel sealing. Finally, a fine nozzle decreases the throughput of the bioprinting, which is of substantial importance in food biomanufacturing. The application of the static mixer disclosed herein resolves these important challenges. Subsequently, a coaxial microfluidic device was implemented to extrude and form hydrogel fibers from the mixture of the hydrogel precursor, controlling the diameter of the fiber and the orientation of internal microfilaments.

Alginate and GelMA were selected to form the components of the bioink in the proposed biofabrication technique. The bioink was designed based on the synergistic interplay of these two materials, in which each hydrogel plays crucial roles for addressing the requirements of a suitable bioink. Using the biofabrication method, fibers with internal microstructure consisting of consecutive microfilaments of GelMA and alginate were formed. Within the microstructure, the GelMA filaments provided a cell-permissive environment while the stiffer, non-cell-permissive alginate sections provided topological and mechanical cues for cell alignment. A real-time control mechanism over the direction of cellular orientation within the individual fibers can comprise controlling the alignment of microfilaments within the hydrogel fiber through manipulation of flow rates in coaxial microchannels. This feature enables continuous bioprinting of cell-laden constructs with in situ controlled cellular organization.

The biofabrication strategy properly supports cellular activity within the scaffold, in contrast to the hybrid scaffolds fabricated with homogeneously mixed alginate/GelMA hybrid bioink. This is an important outcome since several efforts have been made to harness the printability of the alginate and cell-permissibility of the GelMA by application of their hybrid hydrogels, though, the internal cell spreading and alignment were limited. This issue could not be resolved even by introduction of microfilaments inside fiber using a similar static mixer used in this study. This is due to the presence of alginate within the structure which prevents degradation, and therefore spreading and proliferation, of the encapsulated cells. Micro-compartmentalization in the structure can resolve this problem. Cells can spread and proliferate in the GelMA sections while alginate provides a matrix that allows a printable scaffold with high fidelity.

The multicompartmental hydrogel fibers support cellular maturation toward muscle tissue engineering. The biofabricated hydrogel fibers with internal microfilaments along the fiber axis provide the opportunity for improved mimicking of native muscular tissues and direct myoblast alignment. Staining and gene expression analysis confirmed high potential of the multicompartmental hydrogel fiber for myogenesis. Fascicle-like constructs with densely packed, highly aligned cellular organization were formed, expressing genes associated with myofiber maturation.

### Methods and Materials of Example 1

Materials

Sodium alginate (medium viscosity), calcium chloride (CaCl2), type-A gelatin from porcine skin, methacrylic anhydride (MA), and DAPI were purchased from Sigma-Aldrich. Irgacure 2959 (CIBA Chemicals) was used as photoinitiator (PO. Dulbecco's phosphate buffer saline (DPBS, Gibco), Hank's Balanced Salt Solution (HBSS, Gibco) without calcium and magnesium, Dulbecco's modified eagle medium (DMEM, Gibco), fetal bovine serum (FBS, Gibco), horse serum (Gibco), and penicillin/streptomycin (Gibco) were used for experiments with the cells. Alexa Fluor 488 Phalloidin (Life Technologies) was used for characterization of cells' morphology, while metabolic activity of the cells was examined using PrestoBlue cell viability assay (Invitrogen).

Hydrogels Preparation

GelMA was prepared according to the well-established protocol56 with some modification. Briefly, a 10% solution of gelatin (in DPBS) was prepared by stirring for 1 h at 50° C. Subsequently, 50 μL of MA per 1 g of gelatin was added to the mixture slowly and stirred for 3 h at 50° C. and 250 rpm to perform the methacrylation. To stop the reaction, DPBS was added (5:1 ratio of DPBS:GelMA) and dialysis was performed at 40° C. for 5 days using 12-14 kDa MWCO tubing (Fisher Scientific). Finally, the solution was filtered, frozen at −80° C. for 2 days, and lyophilized for 5 days. GelMA precursor was prepared by mixing 2% PI and 10% GelMA solutions in HBSS with a 1:5 volumetric ratio. The alginate precursor was prepared at a 2% concentration in HBSS.

Biofabrication of Multicompartmental Hydrogel Fibers

The biofabrication was performed either through bioprinting or wetspinning of multicompartmental hydrogel. In both cases, a static mixer integrated with a coaxial microfluidic device was used as the nozzle. The static mixer was prepared by fitting a specific number of 3D printed Kenics helical elements (66:100:4 ratio of diameter:length:thickness of each element) into a barrel with a conical outlet. The barrel was then sealed with a PDMS plug with two openings for hydrogel injection. The microfluidic device for coaxial flow was fabricated by assembling blunt needles with different gauge sizes ([14G and 18G] or [19G and 24G]). The needles were trimmed to such a size that the tip of the inner needle was located at ˜1 mm from the opening of the outer one. Finally, the microfluidic device was attached to the conical static mixer tip. For experiments with cells, the device was incubated in ethanol (70%) followed by washing with autoclaved distilled water three times. To accurately adjust the flow rates of hydrogel and CaCl2 solutions, the inlets were connected to syringes using Tygon tubing (Cole-Parmer), and the flows were controlled using syringe pumps (PHD 2000, Harvard Apparatus). Unless otherwise stated, the flow rates of alginate, GelMA, and CaCl2 solutions were set to 1x, 1x, and 2x, in which the x for bioprinting and wetspinning experiments were set to 10 μL/min and 500 μL/min, respectively. In bioprinting experiments, the setup was mounted on the printing head of the bioprinter (Allevi 3). While the flowrates were controlled using separate syringe pumps, the displacement of the nozzle was controlled by the bioprinter. For wetspinning, the nozzle was placed into a CaCl2 bath at 10° C. while the solutions were extruded. A 2% (w/v) CaCl2 solution was used for ionic gelation of alginate followed by 30 s UV crosslinking of the GelMA using a 365 nm/850 mW source, placed at a distance of 7 cm from the fibers.

Fluid Flow Characterization and Hydrogel Fiber Topography

Finite element simulations were conducted to evaluate the function of the static mixer and flow-focusing device and to examine the mechanism of highly-aligned fibrillar structure formation within the hydrogel fiber. The model was implemented in COMSOL Multiphysics 5 using “Laminar Flow” and “Particle Tracing for Fluid Flow” interfaces. First, a 3D model was designed with the dimensions matching the dimensions of actual static mixer and co-axial microfluidic device. The “Laminar Flow” was then used to simulate the flow of hydrogel and CaCl₂ solutions in the channels through solving the Naiver Stocks equations. Different flow rates were applied to the hydrogel and CaCl₂ inlets in different simulations, corresponding to the experimental flowrates mentioned in the previous section, while the relative pressure was always set to zero at the outlet. All boundaries were considered to have a “non-slip” condition and the model was discretized with fine free tetrahedral elements. Finally, the model was solved using a “Stationary Solver”. To evaluate the pressure inside the channels, the pressure obtained through solving the Naiver Stocks equations (the relative pressure, p=pabs−pref, in which pabs is the absolute pressure and pref is the sea-level pressure) was used. Additionally, the shear stress was calculated post simulation through multiplication of shear rate (spf.sr) by the fluid viscosity (spf.mu). The maximum pressure Pmax and the maximum shear stress τmax was determined from the highest values in the simulation domains, provided by the software. The maximum pressure generally happens at the channel entrance, since it is dependent on resistance against the flow, while the maximum shear stress usually happens at the fluid/wall interface, where the channel cross-section area is minimum, because it is proportional to the rate of velocity changes. To track the streams of the hydrogels in the static mixer and for cross section profile, the “Particle Tracing for Fluid Flow” interfaces was used to simulate the movement of 10⁴ massless particles in the previously solved velocity field using a “Time Dependent Solver”. For particle tracing, a “Freeze” boundary condition was set to the channels' walls. To visualize the fiber cross section in the simulations, a “Poincare Map” was implemented with different colors used for the particles injected from different inlets.

Experimentally, fluorescent particles were used to evaluate the cross-sectional profiles of the fibers. After fabrication, fibers were embedded into 3% agarose gel and sliced using a surgical blade. To evaluate the formation of GelMA microfilaments in the alginate matrix, phase contrast microscopy was performed on a Zeiss Observer.D1 microscope. The diameter of final fibers was measured using ZEN 2 software.

Cell Culture

Murine myoblast cell line C2Cl2 (ATCC) was cultured in DMEM supplemented with 10% (v/v) FBS and 1% (v/v) penicillin/streptomycin (culture medium). Cells were incubated at 37° C. in a humidified 5% CO₂ atmosphere and subcultured at 80%-90% confluence. Cell passages 6-8 were utilized for experiments.

For the encapsulation of C2Cl2 myoblasts, cells were trypsinized and detached followed by resuspension in culture medium with the density of 20×106 cells/mL. The solution was then added to GelMA precursor with the volumetric ratio of 1:20 and mixed. Subsequently, cell-laden multicompartmental hydrogel fibers were formed as previously described. After fabrication, the fibers were incubated in the culture medium for future analysis. For evaluating maturation of the myoblasts in the scaffolds, culture medium was replaced with differentiation medium two days after biofabrication. The differentiation medium which prepared using DMEM supplemented with 2% (v/v) horse serum and 1% (v/v) penicillin/streptomycin was replaced every 48 h.

Cellular Morphology Characterization

F-actin/DAPI staining was employed for characterization of cells' morphology. The staining was conducted at room temperature, and HBSS was used for washing steps and solution preparation. Samples were fixed using 4% paraformaldehyde (Electron Microscopy Sciences) for 30 min, washed 3 times, and stained using phalloidin and DAPI as described in the manufacturer's manual, with small modifications. Briefly, cells were permeabilized using 0.2% (v/v) Triton X-100 (Sigma) for 10 min, washed 3 times, followed by blocking with 1% (w/v) bovine serum albumin (Sigma). The samples were then incubated for 40 min in phalloidin (1.65 μM) protected from the light, and subsequently washed 3 times. Nuclei of the cells were then stained using DAPI solution (5 μg/mL) for 15 min, and finally the samples were washed three times. Fluorescence microscopy was performed on the Zeiss Observer.D1 microscope employing an X-Cite 120Q fluorescence source. Subsequently, quantitative analysis of the cellular orientation was performed using Directionality or OrientationJ plugins of FIJI open source software. 57

Determination of Metabolic Activity

Metabolic activity of the encapsulated myoblasts within the hydrogel fiber constructs was measured using a PrestoBlue viability assay. For this purpose, the fabricated hydrogel fibers were cut into smaller segments (˜1 cm) and incubated with 10% PrestoBlue solution (v/v in culture medium) at 37° C. After 1 hr, the solution was collected in a 96 well plate and its fluorescent intensity (550 ex/600 em) was measured using a plate reader (Synergy 2, BioTek). The evaluation was performed 1, 3, 5 and 7 days after fiber fabrication. The background intensity (corresponding to wells with 10% PrestoBlue solution, excluding cell laden fibers) was subtracted and the results were normalized with respect to the values of day 1.

Reverse Transcription Quantitative Polymerase Chain Reaction

Expression levels of three myoblast differentiation genes (MyoD, MRF4 and Myh1) were evaluated using reverse transcription quantitative PCR, RT-qPCR, after 0, 1, 3, 5, 7 and 11 days of fiber fabrication. Total RNA was extracted using RNeasy Plus mini kit (Qiagen) and 1 μg of extracted RNA was reverse-transcribed using QuantiTect Reverse Transcription kit (Qiagen) according to manufacturer's protocol. Real-time PCR was performed on a Rotor-Gene Q (Qiagen) using 2 μL of cDNA template, 2 μL of primer set and 16 μL of SYBR Green Master Mix (Fermentas). Thermal cycle conditions were 10 min denaturation at 95° C. followed by 45 cycles of 10 s at 95° C., 30 s at 60° C. and 30 s at 72° C. The results were normalized to that of GAPDH as reference housekeeping gene and then to the results of day 0 using 2^{-ΔΔCt} method.

Biomimetic incorporation of hollow channels and vasculature is crucial for biofabrication of large tissue engineering constructs. Current technologies are limited in their effectiveness in the fabrication of channels with diameters smaller than hundreds of micrometers. Using the multicompartmental bioprinting system 10 disclosed herein, the co-extrusion of cell-laden hydrogels and sacrificial materials through the system 10 with designed number of helical elements enables one-step fabrication of hydrogel filaments embedding dozens of hollow microchannels (FIG. 17A). The mixer 12 can induce the generation of intercalated layers of the print fluids as the print fluids are continuously extruded through the system 10. The biofabrication of hydrogel filaments, with a diameter of ˜1 mm, containing up to 31 intrinsic hollow channels with sizes comparable to cell dimensions can be formed using the system and methods disclosed herein. Scanning electron microscopy micrographs demonstrates that channels with widths from 100 μm to 22 μm can be produced using a mixer 12 with 3 to 6 helical mixing elements 22. Endothelial cells can be supplemented with sacrificial or crosslinkable biomaterial to accelerate the formation of vasculature. Overall, the system 10 presents an effective and practical tool for the fabrication of pre-vascularized engineered tissues.

The physiological process of angiogenesis within thick and large-sized implants is time-consuming, which results in the failure of clinically sized implants due to massive starvation-induced cell death within the implant. Three-dimensional (3D) scaffolds fabricated with multicompartmental bioprinting system 10 that incorporate hollow microchannels can induce faster angiogenesis in vivo due to the microchannels.

### EXEMPLARY ASPECTS AND ADVANTAGES OF EMBODIMENTS DISCLOSED HEREIN

Advantages of one or more embodiments of the disclosed multicompartmental bioprinting system can include:

The ability to print multiple materials from a single printer (optionally, simultaneously);

The formation of filaments with the controlled spatial pattern of different compartments with cell-scale resolution;

Fabrication of scaffolds in which every single filament creates a biomimetic microenvironment with embedded capillaries (e.g., microcapillaries) and designed physical and chemical properties;

Spatial control of the multiple materials interaction within a filament; and

Printing different internal compartments with adjustable size, architecture, and level of mixing.

The present disclosure provides an advantageous system and method for formation of microtopographies inside a bioprinted scaffold with desired resolution. This system and method can be low cost, can involve one step, and can be robust. The feature size can be decreased down to the cell size, which can therefore be used for controlling cellular behavior. The resolution is not shear- or pressure dependent, and therefore the method does not negatively affect the viability or behavior of the cells. The advantages of different hydrogels can be integrated into a single hydrogel fiber to be printed, toward micro-modulation of cellular behavior, mechanical properties, and morphology of the biofabricated scaffold. The disclosed systems and methods can be applied to most hydrogels and polymers. The disclosed systems and methods can allow the co-printing of multiple cells, molecules, proteins, and other biological or food grade materials, for example, for meat agriculture with desired fat content by co-encapsulation of muscle progenitors and adipocytes.

In non-limiting embodiments, the disclosed systems and methods can be utilized in a variety of industries, including healthcare, and food manufacturing, among others. In one embodiment, the disclosed systems and methods described herein can be used in the food industry, for example, to meet agriculture requirements, since the formed structures enable muscle cell alignment and therefore improves differentiation and maturation. Accelerated maturation can reduce the costs of prolonged culture and differentiation of cells for large-scale meat production. The strategy can allow the introduction of reinforcing filaments for improved meat texture and sensory. Furthermore, different tissues such as fat, muscle and vasculature can be integrated with biomimetic structure to mimic the sensory of the produced meat. The versatility of the system 10 enable biomanufacturing of the meat with desired muscle, fat, and/or vasculature content. The versatility further enable biomanufacturing of meat corresponding to different species or different cuts.

In another embodiment, the disclosed systems and methods can be used in biomedical applications in tissue engineering and regenerative medicine. Controlling cellular organization not only accelerates the maturation of the tissue, but can also offer improved functional recovery of the tissue. For example, in muscle tissue engineering, and enhanced myogenesis can be obtained due to cellular alignment, and the final healed muscle allow higher force generation. Thus, potential users can include, but are not limited to, researchers, surgeons, healthcare providers, and food manufacturing industries.

In another embodiment, the disclosed systems and methods can be used in biomedical applications in engineering of vascularized tissues. The presence of vascular network is crucial for viability of cells encapsulated inside the scaffold, integration of the scaffold with native tissue and accelerated regeneration of the defected tissue.

The disclosed multicompartmental bioprinting system can be used in research and teaching institutions. For research institutions, exemplary users of the disclosed system can include academic researchers (pre-doctoral researchers, post-doctoral researchers, principal investigators, etc.). The multicompartmental bioprinting system can have applications in biomaterial testing, additive manufacturing, and biomedical research (in vitro, in vivo studies). The applications can include any potential use where a controlled deposition of liquid materials is useful.

For teaching institutions, exemplary users of the disclosed system can include students in laboratory-based courses, lecturing professors, and scientific outreach activists. These individuals can use the device for the additive construction of precursor materials as demonstrations of key educational concepts. It is a low cost system which can be accessible by everyone.

For medical applications, exemplary users of the disclosed multi-material bioprinter can include procedural, wound prep, and wound management care providers. The medical applications can range from public hospitals, private practices, and military health care. The end user can include field medics, paramedics, dermatologists, plastic surgeons, wound care nurses, orthopedic surgeons, cardiothoracic surgeons, etc. The ability to have facile control over the composition and organization of subfeatures within a printed scaffold can increases the number of applications over single-material printing; this increase in versatility has direct applications in tissue interfaces, tissue bonding, tissue regeneration, and tissue reconstruction.

### Computing Device

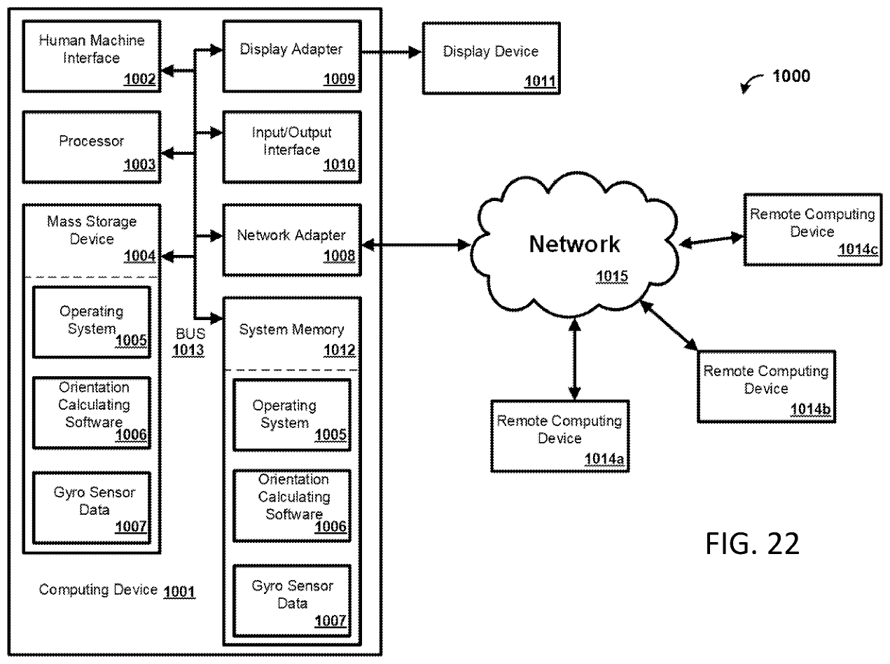

FIG. 22 shows an operating environment 1000 including an exemplary configuration of the computing device 1001 that can be used with the system 10. Further, elements of the computing device can be integral to the printer 10. Still further, elements of the printer 10, such as, for example, the microcontroller 56 (FIG. 6), can be configured as disclosed herein with reference to the computing device 1001. The computing device 1001 may comprise one or more processors 1003, a system memory 1012, and a bus 1013 that couples various components of the computing device 1001, including the one or more processors 1003, to the system memory 1012. In the case of multiple processors 1003, the computing device 1001 may utilize parallel computing.

The bus 1013 may comprise one or more of several possible types of bus structures, such as a memory bus, memory controller, a peripheral bus, an accelerated graphics port, and a processor or local bus using any of a variety of bus architectures.

The computing device 1001 may operate on and/or comprise a variety of computer readable media (e.g., non-transitory). Computer readable media may be any available media that is accessible by the computing device 1001 and comprises, non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as printing data 1007 and/or program modules such as operating system 1005 and orientation calculating software 1006 that are accessible to and/or are operated on by the one or more processors 1003.

The computing device 1001 may also comprise other removable/non-removable, volatile/non-volatile computer storage media. The mass storage device 1004 may provide non-volatile storage of computer code, computer readable instructions, data structures, program modules, and other data for the computing device 1001. The mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1004. An operating system 1005 and print analyzing software 1006 may be stored on the mass storage device 1004. One or more of the operating system 1005 and print analyzing software 1006 (or some combination thereof) may comprise program modules and the print analyzing software 1006. The printing data 1007 may also be stored on the mass storage device 1004. The printing data 1007 may be stored in any of one or more databases known in the art. The databases may be centralized or distributed across multiple locations within the network 1015.

A user may enter commands and information into the computing device 1001 using an input device (not shown). Such input devices comprise, but are not limited to, a keyboard, pointing device (e.g., a computer mouse, remote control), a microphone, a joystick, a scanner, a touchscreen, tactile input devices such as gloves, and other body coverings, motion sensor, and the like. These and other input devices may be connected to the one or more processors 1003 using a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display device 1011 may also be connected to the bus 1013 using an interface, such as a display adapter 1009. It is contemplated that the computing device 1001 may have more than one display adapter 1009 and the computing device 1001 may have more than one display device 1011. A display device 1011 may be a monitor, an LCD (Liquid Crystal Display), light emitting diode (LED) display, television, smart lens, smart glass, and/or a projector. In addition to the display device 1011, other output peripheral devices may comprise components such as speakers (not shown) and a printer (not shown) which may be connected to the computing device 1001 using Input/Output Interface 1010. Any step and/or result of the methods may be output (or caused to be output) in any form to an output device. Such output may be any form of visual representation, including, but not limited to, textual, graphical, animation, audio, tactile, and the like. The display 1011 and computing device 1001 may be part of one device, or separate devices.

The computing device 1001 may operate in a networked environment using logical connections to one or more remote computing devices 1014a,b,c. A remote computing device 1014a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computing device 1001 and a remote computing device 1014a,b,c may be made using a network 1015, such as a local area network (LAN) and/or a general wide area network (WAN). Such network connections may be through a network adapter 1008. A network adapter 1008 may be implemented in both wired and wireless environments. Such networking environments are conventional and commonplace in dwellings, offices, enterprise-wide computer networks, intranets, and the Internet. It is contemplated that the remote computing devices 1014a,b,c can optionally have some or all of the components disclosed as being part of computing device 1001. In further optional aspects, the remote computing device 1014b can be a server that receives and stores logged data from the printer. In optional aspects, some or all data processing can be performed via cloud computing on a computing device or system that is remote to the computing device 1001.

Application programs and other executable program components such as the operating system 1005 are shown herein as discrete blocks, although it is recognized that such programs and components may reside at various times in different storage components of the computing device 1001, and are executed by the one or more processors 1003 of the computing device 1001. An implementation of print analyzing software 1006 may be stored on or sent across some form of computer readable media. Any of the disclosed methods may be performed by processor-executable instructions embodied on computer readable media.

### EXEMPLARY ASPECTS

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1A: A multicompartmental bioprinting system comprising:
a connector, which is delivering different biomaterials to the mixer,
a mixer, which is forming streams of the mixture of different biomaterials with desired spatial distribution along and across the printing fiber;
a nozzle in communication with the mixer, wherein the nozzle defines the cross-sectional geometry of the final printed fiber;
a coaxial fluidic system, which can deliver crosslinking agent to form the fiber, control the size of the fiber, and control the organization of the internal fiber's compartments.
wherein the multicompartmental bioprinting system is configured to simultaneously or sequentially extrude the first, second, and/or additional printable materials from the outlet.

Aspect 2A: The multicompartmental bioprinting system of aspect 1A, wherein the multi-material printer is capable of extruding, mixing, and crosslinking materials of varying chemical, structural, and physical properties that can be mixtures of materials, cells, growth factors, nucleic acids, lipids, and other biomaterials.

Aspect 3A: The multicompartmental bioprinting system of aspect 1A, wherein one or more of the first, second, and/or additional printable materials are configured to be removed from the extruded filament or subfilament to form capillaries, channels or cavities.

Aspect 4A: The multicompartmental bioprinting system of aspect 1A, wherein the biomaterials can interact to crosslink, form secondary structures, foam, gas, cause destructive or constructive interference, etc.

Aspect 5A: The multicompartmental bioprinting system of aspect 1A, further comprising a mixer that is configured to mix the respective first, second, and/or subsequent materials from the first, second, and/or additional containers

Aspect 6A: The multicompartmental bioprinting system of aspect 5A, wherein the mixer comprises at least one mixing element that can generate lengthwise or cross sectional features including different mixing ratios, linear compartments, coaxial compartments, multicompartmental geometries, gradients, or any combination of the aforementioned conditions of different biomaterials.

Aspect 7A: The multicompartmental bioprinting system of aspect 6A, wherein at least one helical mixing element comprises a plurality of helical mixing elements that are longitudinally spaced along the mixer, wherein adjacent mixing elements of the plurality of mixing elements are rotationally offset from each other.

Aspect 8A: The multicompartmental bioprinting system of aspect 5A, wherein the bioprinter is configured to print a filament or subunit of a larger tissue construct such as a drop comprising a plurality of linear or radial compartments.

Aspect 9A: The multicompartmental bioprinting system of aspect 5A, wherein the mixer is a Christmas-tree gradient generator-style mixer.

Aspect 10A: The multicompartmental bioprinting system of aspect 5A, wherein the mixer and nozzle are unitarily formed as a monolithic component.

Aspect 11A: The multicompartmental bioprinting system of aspect 5A, wherein the mixer and nozzle is configured to print internal sub-filament structures for the creation of vasculature, nerves, or lymphatic systems.

Aspect 12A: The multicompartmental bioprinting system aspect 1A, further comprising of a fluidic channel that is configured to expose chemical, ionic, physical, or enzymatic agents at any point of the extrusion process to aid in disinfection, crosslinking, generation of internal or external geometries, or other features.

Aspect 13A: The multicompartmental bioprinting system aspect 1A, further comprising of a source that is configured to heat the air around the extruded filament or subunit at the point of extrusion process to aid in crosslinking, reduction in contamination, removal of temperature-sensitive materials, etc.

Aspect 14A: The multicompartmental bioprinting system aspect 1A, where the fibers can be printed in a support bath or other environmental conditions like vapor to aid physical support, structural complexity, crosslinking, or some other feature of the extrusion process.

Aspect 15A: The multicompartmental bioprinting system aspect 1A, wherein the nozzle has any geometric shape and array of shapes.

Aspect 16A: A method comprising:
extruding, first, second, and/or additional biomaterials;
mixing, with a mixer, the first, second, and/or additional biomaterials to form a mixture having a predetermined cross sectional structure and composition;
extruding, through a nozzle, the mixture to form a filament or subunit such as a drop;
depositing the fiber upon exposure to a sheath flow for crosslinking, controlling the fiber dimension, and controlling the organization of internal compartment; and
where the system or deposition of the multicompartmental material is controlled manually or automatically via direct or indirect means.

Aspect 17A: The method of aspect 16A, wherein depositing the mixture to form the filament or subunit of a scaffold comprises depositing the mixture for treating a defect or injury in musculoskeletal, skin, bone, eye, mucosal, heart, liver tissues, or reconstructing new tissues for cosmetic or non-cosmetic purposes in a subject in need thereof, comprising: using a printer to deposit printing materials outside or inside the defect of the subject (patient, animal, etc).

Aspect 18A: The method of aspect 16A, wherein depositing the mixture to form the filament or subunit of a scaffold comprises depositing the mixture for formation of edible products, for example, in food biomanufacturing through cellular agriculture, plant-based foods and fermentation-based products.

Aspect 19A: The method of aspect 16A, wherein the predetermined cross sectional structure comprises linear or radial striations of the first, second, and/or subsequent materials.

Aspect 20A: The method of aspect 16A, wherein the mixer generates different cross sectional mixing ratios, linear compartments, coaxial compartments, multicompartmental geometries, gradients, etc. of the biomaterials.

Aspect 21A: The method of aspect 16A, wherein the extrusion of each biomaterial can be controlled individually or simultaneously at constant or varying rates.

Aspect 21A: The method of aspect 16A, wherein the printing materials comprises at least one of a polymer, a protein, nucleic acids, lipids, ceramic, mixture, or other biomaterials wherein printing material comprises any polymer such as polycaprolactone, polylactic acid, poly glucolic acid and their co-polymers, alginate, chitosan, etc, proteins for example collagen or gelatin, etc and their mixtures. The polymers, proteins or their mixtures can be functionalized with different chemicals and chemistries. The polymers or their mixture or proteins and their mixtures can be mixed with nanoparticles of any shape or microparticles of any shape made or chemicals made from any material composition examples are metal (silver, gold, magnesium, zinc, selenium, etc), metal oxides, metal peroxides, bioglasses, radiopaque agents, antibacterial compounds and agents, antimicrobial compounds and agents, antibiotics, bioceramics, ceramics, oxygen generating materials, crosslinking agents, proteins, vitamins, lipids, phospholipids, fatty acids, biological factors, polysaccharides, nucleic acids, growth factors, hydroxyapetite, calcium phosphate, carbon nanotubes, quaternary ammonium compounds, graphene, graphene oxide, carbon derived materials, liquid crystals, peptides, chitosan, silver nitride, platelet rich plasma, bone marrow-derived materials, pain killers, anti-inflammatory drugs or reagents, blood-derived materials and their combinations, etc. The concentrations of the nanoparticles or microparticles or chemicals can be having any range.

Aspect 22A: The method of aspect 17A, wherein the filament or subunit is constructed in vitro or deposited into a tissue defect, wherein the tissue defect has any dimension and depth.

Aspect 23A: The method of aspect 17A, wherein the filament or subunit is constructed in vitro or deposited into a tissue defect, wherein the tissue defect has an irregular shape and involves multiple tissues.

Aspect 24A: The method of aspect 16A, wherein the extruded filament comprises two or multiple different cell types, growth factors mixed or placed in distinct regions.

Aspect 25A: The method of aspect 16A, wherein the extruded filament comprises two or multiple different cell types, growth factors mixed or placed in distinct regions to form vasculatular, neural, or lymphatic systems.

Aspect 26A: The method of aspect 16A, wherein the extruded filament comprises a sacrificial component to form channels or conduits during or after printing.

Aspect 27A: The method of aspect 16A, wherein one of the biomaterials is sacrificed to form internal capillaries and structures for the generation of cell-scale cues; construction of other features like vasculature, nerve, or lymph systems; etc.

Aspect 28A: The method of aspect 16A, wherein a physical, radiative, chemical, ionic, and/or enzymatic crosslinking mechanism is used to crosslink at least one of the biomaterials at any point during or after the extrusion process.

Aspect 29A: The method of aspect 16A, wherein emitted chemical, ionic, physical, or enzymatic agents at any point of the extrusion process can aid in disinfection, crosslinking, generation of internal or external geometries, or other features.

Aspect 30A: The method of aspect 16A, wherein a source that is configured to heat the air around the extruded filament or subunit at the point of extrusion process to aid in crosslinking, reduction in contamination, removal of temperature-sensitive materials, etc.

Aspect 31A: The method of aspect 16A, wherein the nozzle can be printed in a support bath to aid physical support, structural complexity, crosslinking, or some other feature of the extrusion process.

Aspect 1B: A (bio)printing system comprising:
a mixer having a longitudinal axis, the mixer defining a flow channel that extends along the longitudinal axis, the mixer is comprising:
at least one inlet configured to receive a first printable biomaterial and a second printable biomaterial;
an outlet spaced from the inlet along the longitudinal axis; and
at least one mixing element positioned within the flow channel between the at least one inlet and the outlet of the mixer, wherein the at least one mixing element is configured to adjust the spatial distribution of different printable materials across and along the longitudinal axis.
The system can include a connector delivering the biomaterials to the mixer, a nozzle with a desired outlet shape that is configured to receive the mixture from the mixer, and a coaxial fluidic channel that crosslinks at least one of the printable biomaterials and controls the fiber diameter and the organization of internal compartments inside the mixture.

Aspect 2B: The system of aspect 1B, wherein the bioprinter is configured to print a fiber comprising a plurality of linear, angled or radial compartments.

Aspect 3B: The system of aspect 1B, wherein the bioprinter is configured to print droplets comprising a plurality of spherical, spherical wedges, linear, angled or radial compartments.

Aspect 4B: The system of aspect 1B, wherein the bioprinter is configured to print a fiber comprising a gradient of different materials across or along the fiber.

Aspect 5B: The system of aspect 1B, wherein the bioprinter is configured to print a fiber comprising internal longitudinal compartments of desired shape.

Aspect 6B: The system of aspect 1B, wherein the bioprinter is configured to print a fiber with nonhomogeneous mixture of two or more different materials with varied relative concentration.

Aspect 7B: The system aspect 1B, wherein the mixer is a Christmas-tree gradient generator-style mixer.

Aspect 8B: The system of aspect 1B, wherein the mixer and nozzle are unitarily formed as a monolithic component.

Aspect 9B: The system of aspect 1B, wherein a nozzle with desired outlet shape defines the cross-sectional profile of the printing fiber.

Aspect 10B: The system of aspect 1B, wherein a coaxial fluidic system crosslinks at least one of the printable materials, control the fiber size and organization of the internal components.

Aspect 11B: The system of aspect 1B, wherein the at least one helical mixing element comprises a plurality of helical mixing elements that are arranged along the longitudinal axis of the mixer.

Aspect 12B: The system of aspect 2B, wherein each mixing element of the plurality of mixing element is rotationally offset from each adjacent mixing element of the plurality of mixing elements.

Aspect 13B: The system of aspect 2B, wherein the plurality of mixing elements comprises from three mixing elements to eight mixing elements.

Aspect 14B: The system of aspect 1B, wherein the mixer defines a taper along the longitudinal axis toward the outlet.

Aspect 15B: The system of aspect 1B, wherein the at least one inlet comprises a first inlet that is configured to receive the first printable biomaterial and a second inlet that is configured to receive the second printable biomaterial.

Aspect 16B: The system of aspect 1B, further comprising a sleeve that defines a sheath channel that surrounds the outlet of the nozzle, wherein the sleeve comprises an inlet that is configured to receive a crosslinker.

Aspect 17B: The system of aspect 16B, further comprising:
at least one first actuator that is configured to effect flow of the first printable biomaterial and the second printable biomaterial;
a second actuator that is configured to effect flow of the crosslinker.

Aspect 18B: The system of aspect 16B, wherein the sleeve comprises an outlet that is spaced from the outlet of the mixer along the longitudinal axis of the mixer in a direction away from the at least one inlet of the mixer.

Aspect 19B: The system of aspect 1B, further comprising a supply of the first printable biomaterial and a supply of the second printable biomaterial.

Aspect 20B: A method comprising:
extruding, through a static mixer, a first printable biomaterial and a second printable biomaterial (or more printable biomaterials) to form a mixture having a predetermined cross sectional structure, wherein the mixer comprises defines a flow channel that extends along the longitudinal axis, wherein the mixer comprises at least one mixing element positioned within the flow channel.
and depositing, through a nozzle, the mixture to form a fiber or sequence of droplets with predetermined internal structure.

Aspect 21B: The method of aspect 20B, wherein the first printable biomaterial is a sacrificial biomaterial.

Aspect 22B: The method of aspect 21B, wherein the second printable biomaterial is a hydrogel.

Aspect 23B: The method of aspect 20B, wherein the static mixer has an outlet, the method further comprising flowing, through a sheath channel of a sleeve that surrounds the outlet of the static mixer, a crosslinker.

Aspect 24B: The method of aspect 23B, further comprising controlling a flow rate of the crosslinker relative to a cumulative flow rate of the first and second printable biomaterials to control a diameter of the fiber.

Aspect 25B: The method of aspect 24B, wherein the outlet of the nozzle has a diameter, wherein the flow rate of the crosslinker relative to the cumulative flow rate of the first and second printable biomaterials is selected to form a fiber having a diameter that is less than the diameter of the outlet of the static mixer.

Aspect 26B: The method of aspect 23B, wherein extruding, through the static mixer, the first and second printable biomaterials forms longitudinally extending striations, wherein the flow rate of the crosslinker relative to the cumulative flow rate of the first and second printable biomaterials is selected to deviate at least a portion of the longitudinally extending striations in a radial direction

Aspect 27B: The method of aspect 23B, wherein at least one of the first printable biomaterial or the second printable biomaterial comprises living cells.

Aspect 28B: The method of aspect 23B, further comprising irradiating the fiber to crosslink at least one of the first printable biomaterial or the second printable biomaterial.

Aspect 29B: The method of aspect 23B, further comprising applying at least one biotextile technique to the fiber with at least one additional fiber to form a biomimetic assembly of multicompartmental hydrogel fibers, wherein the at least one biotextile technique comprises: (i) weaving, (ii) braiding, (iii) knotting, (iv) coil formation, or a combination thereof.

Aspect 1C: A system comprising:
a mixer having a longitudinal axis, the mixer defining a flow channel that extends along the longitudinal axis, the mixer comprising:
at least one inlet configured to receive a first printable biomaterial and a second printable biomaterial;
an outlet spaced from the inlet along the longitudinal axis; and
at least one mixing element positioned within the flow channel between the at least one inlet and the outlet of the mixer, wherein the at least one mixing element is configured to control a spatial distribution of the first and second printable biomaterials across and along the longitudinal axis.

Aspect 2C: The system of aspect 1C, wherein each mixing element of the at least one mixing element is a helical mixing element that is configured to divide the flow channel into opposed flow channel segments.

Aspect 3C: The system of aspect 2C, wherein the at least one helical mixing element comprises a plurality of helical mixing elements that are arranged along the longitudinal axis of the mixer.

Aspect 4C: The system of aspect 3C, wherein each mixing element of the plurality of mixing element is rotationally offset from each adjacent mixing element of the plurality of mixing elements.

Aspect 5C: The system of aspect 3C or aspect 4C, wherein the plurality of mixing elements comprises from three mixing elements to eight mixing elements.

Aspect 6C: The system of any one of aspects 1C-5C, wherein the mixer defines a taper along the longitudinal axis toward the outlet.

Aspect 7C: The system of any one of aspects 1C-6C, wherein the at least one inlet comprises a first inlet that is configured to receive the first printable biomaterial and a second inlet that is configured to receive the second printable biomaterial.

Aspect 8C: The system of any one of aspects 1C-7C, further comprising a sleeve that defines a sheath channel that surrounds the outlet of the mixer, wherein the sleeve comprises an inlet that is configured to receive a crosslinker.

Aspect 9C: The system of aspect 8C, further comprising:
at least one first actuator that is configured to effect flow of the first printable biomaterial and the second printable biomaterial; and
a second actuator that is configured to effect flow of the crosslinker.

Aspect 10C: The system of aspect 8C or aspect 9C, wherein the sleeve comprises an outlet that is spaced from the outlet of the mixer along the longitudinal axis of the mixer in a direction away from the at least one inlet of the mixer.

Aspect 11C: The system of any one of aspects 1C-10C, further comprising a supply of the first printable biomaterial and a supply of the second printable biomaterial, wherein each of the supply of the first printable biomaterial and the supply of the second printable biomaterial is in fluid communication with the mixer.

Aspect 12C: The system of any one of aspects 1C-11C, wherein the bioprinter is configured to print a fiber comprising a plurality of linear, angled or radial compartments.

Aspect 13C: The system of any one of aspects 1C-12C, wherein the bioprinter is configured to print droplets comprising a plurality of spherical, spherical wedges, linear, angled or radial compartments.

Aspect 14C: The system of any one of aspects 1C-13C, wherein the bioprinter is configured to print a fiber comprising a gradient of different materials across or along the fiber.

Aspect 15C: The system of any one of aspects 1C-14C, wherein the bioprinter is configured to print a fiber comprising internal longitudinal compartments of desired shape.

Aspect 16C: The system of any one of aspects 1C-15C, wherein the bioprinter is configured to print a fiber with a nonhomogeneous mixture of the first and second printable biomaterials with varied relative concentration.

Aspect 17C: The system of any one of aspects 1C-16C, wherein the mixer is a Christmas-tree gradient generator-style mixer.

Aspect 18C: The system any one of aspects 1C-17C, wherein the mixer and nozzle are unitarily formed as a monolithic component.

Aspect 19C: The system of any one of aspects 1C-18C, wherein the mixer comprises a nozzle that defines the outlet of the mixer, wherein the outlet has a shape that defines an outer cross-sectional profile of the printing fiber.

Aspect 20C: The system of any one of aspects 1C-19C, wherein a coaxial fluidic system crosslinks at least one of the printable materials, control the fiber size and organization of the internal components.

Aspect 21C: A method of using the device as in any one of the preceding claims, the method comprising:
extruding, through the mixer, the first printable biomaterial and the second printable biomaterial (or more printable biomaterials) to form a mixture having a predetermined cross sectional structure; and
and depositing, through the outlet, the mixture to form a fiber or sequence of droplets with a predetermined internal structure.

Aspect 22C: The method of aspect 21C, wherein the first printable biomaterial is a sacrificial biomaterial.

Aspect 23C: The method of aspect 22C, wherein the second printable biomaterial is a hydrogel.

Aspect 24C: The method of any one of aspects 21C-23C, wherein the static mixer has an outlet, the method further comprising flowing, through a sleeve that surrounds the outlet of the static mixer, a crosslinker.

Aspect 25C: The method of aspect 24C, further comprising controlling a flow rate of the crosslinker relative to a cumulative flow rate of the first and second printable biomaterials to control a diameter of the fiber.

Aspect 26C: The method of aspect 25C, wherein the outlet of the nozzle has a diameter, wherein the flow rate of the crosslinker relative to the cumulative flow rate of the first and second printable biomaterials is selected to form a fiber having a diameter that is less than the diameter of the outlet of the static mixer.

Aspect 27C: The method of any one of aspects 21C-26C, wherein extruding, through the static mixer, the first and second printable biomaterials forms longitudinally extending striations, wherein the flow rate of the crosslinker relative to the cumulative flow rate of the first and second printable biomaterials is selected to deviate at least a portion of the longitudinally extending striations in a radial direction.

Aspect 28C: The method of any one of aspects 21C-27C, wherein at least one of the first printable biomaterial or the second printable biomaterial comprises living cells.

Aspect 29C: The method of any one of aspects 21C-28C, further comprising irradiating the fiber to crosslink at least one of the first printable biomaterial or the second printable biomaterial.

Aspect 30C: The method of any one of aspects 21C-29C, further comprising applying at least one biotextile technique to the fiber with at least one additional fiber to form a biomimetic assembly of multicompartmental hydrogel fibers, wherein the at least one biotextile technique comprises: (i) weaving, (ii) braiding, (iii) knotting, (iv) coil formation, or a combination thereof.

Aspect 31C: The method of any one of aspects 21C-30C, wherein depositing the mixture comprises depositing the mixture for formation of an edible product.

Although the foregoing invention has been described in some detail by way of illustration and example for purposes of clarity of understanding, certain changes and modifications may be practiced within the scope of the appended claims.

What is claimed is:

1. A system comprising:
a mixer having a longitudinal axis, the mixer defining a flow channel that extends along the longitudinal axis, the mixer comprising:
at least one inlet configured to receive a first printable biomaterial and a second printable biomaterial;
an outlet spaced from the inlet along the longitudinal axis; and
at least one mixing element positioned within the flow channel between the at least one inlet and the outlet of the mixer, wherein the at least one mixing element is configured to control a spatial distribution of the first and second printable biomaterials across and along the longitudinal axis.

2. The system of claim 1, wherein each mixing element of the at least one mixing element is a helical mixing element that is configured to divide the flow channel into opposed flow channel segments.

3. The system of claim 2, wherein the at least one helical mixing element comprises a plurality of helical mixing elements that are arranged along the longitudinal axis of the mixer.

4. The system of claim 3, wherein each mixing element of the plurality of mixing element is rotationally offset from each adjacent mixing element of the plurality of mixing elements.

5. (canceled)

6. (canceled)

7. The system of claim 1, wherein the at least one inlet comprises a first inlet that is configured to receive the first printable biomaterial and a second inlet that is configured to receive the second printable biomaterial.

8. The system of claim 1, further comprising a sleeve that defines a sheath channel that surrounds the outlet of the mixer, wherein the sleeve comprises an inlet that is configured to receive a crosslinker.

9. The system of claim 8, further comprising:
at least one first actuator that is configured to effect flow of the first printable biomaterial and the second printable biomaterial; and
a second actuator that is configured to effect flow of the crosslinker.

10. The system of claim 8, wherein the sleeve comprises an outlet that is spaced from the outlet of the mixer along the longitudinal axis of the mixer in a direction away from the at least one inlet of the mixer.

11. (canceled)

12. The system of claim 1, wherein the bioprinter is configured to print a fiber comprising a plurality of linear, angled or radial compartments.

13. The system of claim 1, wherein the bioprinter is configured to print droplets comprising a plurality of spherical, spherical wedges, linear, angled or radial compartments.

14. The system of claim 1, wherein the bioprinter is configured to print a fiber comprising a gradient of different materials across or along the fiber.

15. The system of claim 1, wherein the bioprinter is configured to print a fiber comprising internal longitudinal compartments of desired shape.

16. The system of claim 1, wherein the bioprinter is configured to print a fiber with a nonhomogeneous mixture of the first and second printable biomaterials with varied relative concentration.

17. (canceled)

18. The system of claim 1, wherein the mixer and nozzle are unitarily formed as a monolithic component.

19. (canceled)

20. (canceled)

21. A method of using the device of claim 1, the method comprising:
extruding, through the mixer, the first printable biomaterial and the second printable biomaterial (or more printable biomaterials) to form a mixture having a predetermined cross sectional structure; and
and depositing, through the outlet, the mixture to form a fiber or sequence of droplets with a predetermined internal structure.

22. The method of claim 21, wherein the first printable biomaterial is a sacrificial biomaterial, and wherein the second printable biomaterial is a hydrogel.

23. (canceled)

24. The method of claim 21, wherein the static mixer has an outlet, the method further comprising:
flowing, through a sheath channel of a sleeve that surrounds the outlet of the static mixer, a crosslinker; and
controlling a flow rate of the crosslinker relative to a cumulative flow rate of the first and second printable biomaterials to control a diameter of the fiber,
wherein the outlet of the nozzle has a diameter, wherein the flow rate of the crosslinker relative to the cumulative flow rate of the first and second printable biomaterials is selected to form a fiber having a diameter that is less than the diameter of the outlet of the static mixer.

25. (canceled)

26. (canceled)

27. The method of claim 21, wherein extruding, through the static mixer, the first and second printable biomaterials forms longitudinally extending striations, wherein the flow rate of the crosslinker relative to the cumulative flow rate of the first and second printable biomaterials is selected to deviate at least a portion of the longitudinally extending striations in a radial direction.

28. (canceled)

29. The method of claim 21, further comprising irradiating the fiber to crosslink at least one of the first printable biomaterial or the second printable biomaterial.

30. The method of claim 21, further comprising applying at least one biotextile technique to the fiber with at least one additional fiber to form a biomimetic assembly of multicompartmental hydrogel fibers, wherein the at least one biotextile technique comprises: (i) weaving, (ii) braiding, (iii) knotting, (iv) coil formation, or a combination thereof.

31. (canceled)
